(19) United States
(12) Patent Application Publication
(10) Pub. No.: US 2026/0015629 A1
(43) Pub. Date: Jan. 15, 2026

(54) METHODS AND COMPOSITIONS OF MATTER FOR INERT BIOENGINEERING OF A BIOLOGICAL ENTITY

(71) Applicant: INDEX BIOSYSTEMS INC., Toronto (CA)

(72) Inventors: Michael BORG, St. Catharines (CA); Jeremy N. FRIEDBERG, Guelph (CA); David SINGER, Toronto (CA); Alessandra BIZ, York (CA)

(21) Appl. No.: 18/294,635

(22) PCT Filed: Aug. 3, 2022
(86) PCT No.: PCT/CA2022/051181

§ 371 Date: Feb. 2, 2024

Related U.S. Application Data
(60) Provisional application No. 63/228,933, filed on Aug. 3, 2021.

(51) Int. Cl. C12N 15/90; C12N 15/113; C12Q 1/6869; C12Q 1/6888; G16B 35/20

(52) U.S. Cl. CPC C12N 15/902; C12N 15/113; C12Q 1/6869; C12Q 1/6888; G16B 35/20; C12N 2310/20

(57) ABSTRACT

A bioengineering method which comprises introducing an inert nucleic acid cassette into a biological entity without introducing or modifying characteristics or traits in the biological entity. The method comprises receiving or providing a sample comprising the biological entity having a nucleic acid sequence; selecting an integration site in the nucleic acid sequence for inserting the inert nucleic acid cassette; designing the inert cassette with optimized primer sequences, optimized probe sequences, optimized stop codons and disrupted start codons, and inserting the inert nucleic acid cassette into the biological entity at the integration site; and validating that no characteristics have been added or modified in the biological entity.

This application is a national stage application under 35 U.S.C. § 371 of International Application No. PCT/CA2022/051181, filed 3 Aug. 2022, which claims the priority benefit of U.S. Provisional Patent Application Ser. No. 63/228,933, filed 3 Aug. 2021.

The instant application contains a computer readable Sequence Listing which has been submitted electronically in XML format (“Sequence Listing XML”) and is hereby incorporated by reference in its entirety. The Sequence Listing XML, created Jul. 29, 2025, is named 08949105US1 July 29.xml and is 147,955 bytes in size.

### FIELD OF THE INVENTION

The present invention generally relates to the identification and/or tracking of supply chain products and materials. More specifically, the present invention relates to methods and compositions for the inert bioengineering of a biological entity for product and strain identification. The present invention further relates to methods and compositions of a positive control for nucleic acid tests (NAT) via an inert bioengineered biological entity.

### BACKGROUND OF THE INVENTION

The concept of biological identification using DNA barcodes which are native to an organism was first developed by Dr. Hebert at the University of Guelph. These barcodes consist of sequences of nucleotides unique to the organism or biological entity. These breakthrough technologies made it is possible to efficiently detect DNA barcodes in isolated DNA using standard DNA amplification and molecular biology protocols. These barcoding techniques quickly became ubiquitous in various industries and are commonly used in environmental DNA research; and within commercial inspection, verification, testing and certification companies.

A decade after Hebert published his paper in 2003, two patent applications (US 2019/0285602 and US 2017/0021611) were filed relating to the use of synthesized cell-free DNA markers for tagging products throughout supply chains. These DNA markers rely on industrial-scale polymerase chain reaction (PCR) manufacturing systems, and remain prohibitively expensive for large-scale applications. In addition, extracellular or cell-free DNA is unstable when exposed to the extracellular environment, or when added to industrial processes. This instability has prompted research in encapsulation techniques meant to protect the integrity of cell-free DNA fragments. One example uses silica to encapsulate DNA-based tracers. Encapsulated DNA-based tracer solutions continue to have substantial economic limitations relative to standard cell-free DNA, which makes them unsuitable for scalable industrial supply chain adoption.

Thankfully, advancements in biotechnology have created efficiencies such that it is now economically feasible to edit the genomes of almost any organism. Moreover, it is now feasible to develop biologically traceable technologies that leverage the replicative and protective capacity of an organism to identify any biological entity in the supply chain, and/or to function as a proxy for the identification of supply chain products and materials. These technological breakthroughs enabled DNA-based biomarking without relying on expensive industrial PCR or encapsulation approaches. Index Biosystems bioengineered short identifying barcode sequences of DNA into an organism and scale using industrial fermentation bioaccumulation. However, this technology can result in potentially restrictive regulatory treatment since it relies on genetically engineering an organism. As such, it becomes essential to address concerns of risk associated with the bioengineering of the host organism. There is thus need for methods and compositions of matter which enable the introduction of exogenous DNA into a recipient organism or biological entity such that no characteristics are introduced, and no existing characteristics are modified.

### SUMMARY OF THE INVENTION

The shortcomings of the prior art are generally mitigated by the method as described herein for inert bioengineering of a biological entity, which involves introducing an inert nucleic acid cassette into a biological entity without introducing or modifying characteristics or traits in the biological entity.

Accordingly, there is provided herein a method which comprises receiving or providing a sample comprising the biological entity having a nucleic acid sequence, typically a genome. An integration site is selected in the nucleic acid sequence for inserting the inert nucleic acid cassette. Once the site has been selected, the method then comprises designing the inert nucleic acid cassette (i.e. a synthetic nucleic acid) with optimized primer sequences, optimized probe sequences, optimized stop codons and disrupted start codons, and inserting the inert nucleic acid cassette into the biological entity. The following step comprises validating that no characteristics have been added or modified in the biological entity. Optionally, the method may include a step of inactivating the biological entity.

According to a preferred embodiment, selecting an integration site comprises determining if a new site is required. For example, this may include checking a data store with information on the biological material to ascertain if a prior engineering event has occurred, and thus if a new site is required.

According to a further embodiment, selecting an integration site comprises screening the genome of the biological entity and identifying any characterized and/or putative genes, open reading frames and/or features of concern.

According to a further embodiment, the selected integration site is within a predetermined distance in base pairs (bp), preferably at least 250 base pairs (bp), from putative genes, open reading frames and/or features of concern. In a non-limiting embodiment, the predetermined distance is typically between 250-1000 bp from the putative genes, open reading frames and/or features of concern.

According to a further embodiment, the selected integration site is located within heterochromatic regions and/or long terminal repeats (LTRs).

According to a further embodiment, selecting an integration site comprises determining an absence of characterized and/or putative promoters directing transcription of the cassette.

According to a further embodiment, designing the inert cassette comprises creating landing pads for assisting with the integration of the inert cassette at the integration site.

According to a further embodiment, the landing pad is a CRISPR-Cas9 target sequence complementary to an optimized gRNA spacer.

According to a further embodiment, the integration site is selected in silico using databases.

According to a further embodiment, inactivating the biological entity comprises thermal inactivation, chemical inactivation, nutrient deprivation over time, or a combination thereof.

In further non-limiting embodiments, the step of validating that no characteristics have been added or modified in the biological entity may include analyzing the transcriptional profile of the biological entity to validate that transcripts of the inert nucleic acid cassette cannot be detected.

In another non-limiting embodiment, the step of validating that no characteristics have been added or modified in the biological entity may include whole genome sequencing of the transformed biological entity to validate that the inert nucleic acid cassette has been integrated at the selected site, without any off-target effects. Without limitation, off-target effects may include one or more mutations in the cassette or elsewhere in the genome of the biological entity. Also provided herein is an inert nucleic acid cassette for inert bioengineering of a biological entity, the inert nucleic acid cassette comprising optimized primer sequences, optimized probe sequences, optimized stop codons and disrupted start codons.

In an embodiment of the inert nucleic acid cassette, the optimized stop codons may comprise stop codons for all reading frames in order to stop any transcription initiation within the cassette and the sequences flanking the cassette.

In an embodiment of the inert nucleic acid cassette, at least one barcode sequence from at least one organism having a known sequence may be inserted in the inert nucleic acid cassette for detecting the at least one organism in a biological entity. The at least one organism to be detected may be a known pathogen and/or a known allergen. The at least one barcode sequence may further comprise randomized sequences for differentiating the at least one barcode sequence from the sequence of the at least one organism.

Other and further aspects and advantages of the described method will be better understood upon the reading of the illustrative embodiments about to be described or will be indicated in the appended claims, and various advantages not referred to herein will occur to one skilled in the art upon employment of the invention in practice.

### BRIEF DESCRIPTION OF THE DRAWINGS

The aspects, features and advantages of the invention will become more readily apparent from the following description, reference being made to the accompanying drawings in which:

FIG. 1 is a flow chart of a method for inert bioengineering of a biological entity.

FIG. 2 is a flow chart of a method for selecting an appropriate integration site, according to an embodiment.

FIG. 3 is an example of a selected site, according to an embodiment.

FIG. 4 is a flow chart of a method for designing the inert nucleic acid cassette, according to an embodiment.

FIG. 5 is an example of an inert nucleic acid cassette design, according to an embodiment. Shown are the sense (SEQ ID NO: 11) and antisense (SEQ ID NO: 71) strands, as well as the amino acid sequences encoded by the 3 reading frames of the sense (SEQ ID NOs: 72-78, SEQ ID NOs: 78-80, and SEQ ID NOs: 81-83) and antisense (SEQ ID NOs: 84-87, SEQ ID NOs: 88-93, and SEQ ID NOs: 94-98) strands. Also shown are sequences corresponding to the 5′ stop codons (SEQ ID NO: 12); qPCR primer forward (SEQ ID NO: 13), LAMP F3 (SEQ ID NO: 14), LAMP FIP 2 (SEQ ID NO: 15), optimal stop codon configuration (SEQ ID NO: 30), FIP 1 (SEQ ID NO: 16), PROBE_qPCR (SEQ ID NO: 17), BIP 1 (SEQ ID NO: 18), BIP 2 (SEQ ID NO: 19), LAMP B3 (SEQ ID NO: 20), qPCR primer bind (SEQ ID NO: 21), and 3′ stop codons (SEQ ID NO: 22).

FIG. 6 is a flow chart of a method for validating that no characteristics have been added or modified in the recipient organism, according to an embodiment.

FIG. 7 is an example of an RNA transcription validation step, according to an embodiment.

FIG. 8 is an example of growth validation step, according to an embodiment.

FIG. 9 is an example of a sugar consumption validation step, according to an embodiment.

FIG. 10 is a flow chart of a method for inactivating the recipient organism according to an embodiment.

FIG. 11 is an example of thermal inactivation that is validated using trypan blue staining according to an embodiment.

FIG. 12 is an example of an inert nucleic acid cassette design for providing a positive control for the identification of a known pathogen, according to an embodiment. Shown are the sense (SEQ ID NO: 99) and antisense (SEQ ID NO: 100) strands, as well as 2019-nCOV_N2 forward primer (SEQ ID NO: 101) and 2019-nCOV_N2 reverse primer (SEQ ID NO: 102).

FIG. 13 is an example of an inert nucleic acid cassette design comprising randomized sequences for providing a positive control for the identification of a known pathogen, according to an embodiment. Shown are the sense (SEQ ID NO: 103) and antisense (SEQ ID NO: 104) strands, as well as 2019-nCOV_N2 forward primer (SEQ ID NO: 101) and 2019-nCOV_N2 reverse primer (SEQ ID NO: 102).

FIG. 14 is an example of an inert nucleic acid cassette design, according to a further embodiment. Shown are the sense (SEQ ID NO: 29) and antisense (SEQ ID NO: 105) strands, as well as the amino acid sequences encoded by the 3 reading frames of the sense (SEQ ID NOs: 106-110, SEQ ID NOs: 111-113, and SEQ ID NOs: 114-118) and antisense (SEQ ID NOs: 119-124, SEQ ID NOs: 125-131, and SEQ ID NOs: 132-134) strands. Also shown are sequences corresponding to the gRNA binding site (SEQ ID NO: 31), sequencing primer F (SEQ ID NO: 32), F3 (SEQ ID NO: 33), RPA F (SEQ ID NO: 34), qPCR primer F (SEQ ID NO: 35), F2 (SEQ ID NO: 36), stop codons (SEQ ID NO: 30), probe qPCR (SEQ ID NO: 38), F1c (SEQ ID NO: 39), RPA R (SEQ ID NO: 40), qPCR Primer R (SEQ ID NO: 41), B1c (SEQ ID NO: 42), sequencing primer R (SEQ ID NO: 43), B2 (SEQ ID NO: 44), and B3 (SEQ ID NO: 45).

FIG. 15 is an example of a selected site, according to an embodiment. Shown are the sense (SEQ ID NO: 135) and antisense (SEQ ID NO: 136) strands. The selected site is 291 bp downstream of the gene GAL7 (SEQ ID NO: 56) and 428 bp upstream of the gene GAL10 (SEQ ID NO: 58) in the sense strand. Shown is a portion of the GAL7 (SEQ ID NO: 56) amino acid sequence (SEQ ID NO: 137).

FIG. 16 is an example of a selected site, according to an embodiment. Shown are the sense (SEQ ID NO: 138) and antisense (SEQ ID NO: 139) strands. Also shown is a sequence upstream of the selected site (SEQ ID NO: 60), a sequence downstream of the selected site (SEQ ID NO: 63), and the putative promoter sequence (SEQ ID NO: 61).

FIG. 17 is an example of a selected site, according to an embodiment. Shown are the sense (SEQ ID NO: 140) and antisense (SEQ ID NO: 141) strands.

FIG. 18 is an example of an RNA transcription validation step, according to an embodiment.

FIG. 19 is an example of growth validation step, according to an embodiment.

FIG. 20 is an example of a sugar consumption validation step, according to an embodiment.

FIG. 21 is an example of an RNA transcription validation step, according to an embodiment.

FIG. 22 is an example of growth validation step, according to an embodiment.

FIG. 23 is an example of a sugar consumption validation step, according to an embodiment.

FIG. 24 is an example of an RNA transcription validation step, according to an embodiment.

FIG. 25 is an example of growth validation step, according to an embodiment.

FIG. 26 is an example of a sugar consumption validation step, according to an embodiment.

FIG. 27 is a flow chart of the whole genome sequencing validation step, according to an embodiment.

### DETAILED DESCRIPTION

Described herein are methods and compositions for inert bioengineering of a biological entity. It will be appreciated that embodiments and examples are provided for illustrative purposes intended for those skilled in the art, and are not meant to be limiting in any way.

The principles and methodologies for assessing the risk of a modified organism has been internationally ratified under the Cartagena Protocol. The Cartagena Protocol on Biosafety is an international agreement which aims to ensure the safe handling, transport and use of living modified organisms (LMOs) resulting from modern biotechnology that may have adverse effects on biological diversity, also taking into account risks to human health.

It reasonably follows that the design of these barcodes and the subsequent bioengineering events must address risks associated with the points-to-consider within the Cartagena Protocol.

The points-to-consider described by the protocol consist of:
Recipient organism or parental organisms
Donor organism or organisms
Vector
Insert or inserts and/or characteristics of modification
Living modified organism
Detection and identification of the living modified organism
Information relating to the intended use
Receiving environment

The common thread throughout these points-to-consider relates to the introduction of characteristics or the modification of existing characteristics. If one were to, in absolute terms, eliminate risks associated with these points-to-consider it would be achieved by bioengineering an inert nucleic acid cassette into a target site within the recipient organism or biological entity such that no characteristics were introduced and no characteristics were modified.

The terminology used herein is in accordance with definitions set out below.

As used herein the expression “biological entity” refers to any organism such as an individual animal, plant, or single-celled life form and microorganisms such as bacteria, yeast, viruses, and fungi. As used herein the term “characteristics” comprises any expressed traits or attributes of the organism original to the organism and prior to the integration of the inert nucleic acid cassette in the genome of the organism. In this context, biological characteristics include but are not limited to metabolic rates and profile; rate of growth, development and reproduction; lipids, carbohydrates and proteomic profiles; sensitivity or response to the environment and structural organization and homeostasis.

As used herein the expression “phenotypical characterization” refers to the identification of observable characteristics in the bioengineered biological identity relative to a control biological entity.

As used herein the term “site” is used to refer to a location, or locus in the genome or nucleic acid sequence of a biological entity where the cassette will be integrated.

As used herein the term “landing pads” is used to refer to exogenous sequences introduced in the genome or nucleic acid sequence of the biological entity to assist with the integration of the cassette at the selected integration sites.

As used herein the term “cassette” is used to refer to a nucleic acid sequence containing optimized primer and probe sequences and stop codons, and disrupted start codons.

As used herein the term “nucleic acid sequence” is used to refer to all genetic material of an organism including chromosomal DNA and extra-chromosomal nucleic acid such as DNA plasmids, mitochondrial DNA and viral RNA. In certain embodiments, “nucleic acid sequence” will refer to the genomic DNA or RNA sequence of the biological entity.

As used herein the expression “inert nucleic acid cassette” refers to a nucleic acid cassette which are unlikely to be transcribed into mRNA and does not contain open reading frames and putative core promoters.

As used herein the expression “disrupted start codons” refers to start codons which have been mutated such that translation of the corresponding mRNA transcript by the ribosomes cannot take place.

As used herein the expression “prior engineering event” refers to a prior event which modified a biological entity using bioengineering tools.

As used herein the expression “features of concern” refers to features found within nucleic acid sequences which have known functions in terms of gene expression regulation, which include known regulatory sequences.

As used herein the expression “regulatory sequences” refers to any nucleic acid sequence capable of increasing or decreasing the expression of specific genes within an organism such as promoters, enhancers, silencers and CpG sites.

As used herein the expression “inactivating the biological entity” refers to killing the biological entity such that the biological entity can no longer reproduce.

As used herein the expression “barcode sequence” refers to a sequence of nucleotides used to identify an organism or biological entity.

As used herein the expression “chemical equivalency assessment” refer to an analysis to determine if there is a general equivalency of the chemical composition of a bioengineered organism or biological entity with its parental lineage.

As used herein the expression “predetermined threshold” is used to refer to the threshold at which the difference between the expression levels of the inert cassette relative to the wild-type becomes statistically significant.

By “about”, it is meant that the value or the number of nucleic acid can vary by 10% of the recited value.

Provided herein are methods and compositions for inert bioengineering of a biological entity, such that a traceable nucleic acid is introduced into the nucleic acid sequence of the biological entity without introducing or modifying characteristics or traits. In certain embodiments, methods as described herein may make use of a unique inert identifier sequence (also referred to herein as a nucleic acid unique identifier sequence), exogenously introduced (i.e. inserted/integrated) into the genome or nucleic acid sequence of a biological entity, in order to provide for identification and/or traceability of the biological entity itself, and/or materials comprising the biological entity and/or materials produced from the biological entity and containing genomic DNA therefrom. In certain embodiments, strategies as described herein may benefit from the durability and replicative capacity of nucleic acid such as DNA to provide identification and/or traceability. Accordingly, the traceability of the materials may in certain non-limiting applications be utilized in the context of authentication and/or identification of biological materials within supply chains. For example, tracking supply chain products and materials within the following industries are contemplated but not limited thereto: fluid management for oil and gas, electronics, packaging, textiles, mining, food, animal feed, pharmaceuticals, and nutraceuticals which includes biologics, synthetics and supplements. Other applications will be apparent to those of ordinary skill in the art.

### Methods for Inertly Bioengineering a Biological Entity

According to a preferred embodiment, there is provided a method for bioengineering a biological entity by introducing exogenous nucleic acids comprising an inert cassette into the biological entity without introducing or modifying characteristics in the biological entity. The method comprises receiving or providing a sample comprising the biological entity having a nucleic acid sequence; selecting an appropriate integration site in the nucleic acid sequence for insertion of the inert cassette; designing the inert cassette comprising optimized primer sequences, probe sequences and stop codons, and disrupted start codons, and inserting the inert nucleic acid cassette into the biological entity at the selected integration site; validating that no characteristics have been added or modified in the biological entity; and inactivating the biological entity.

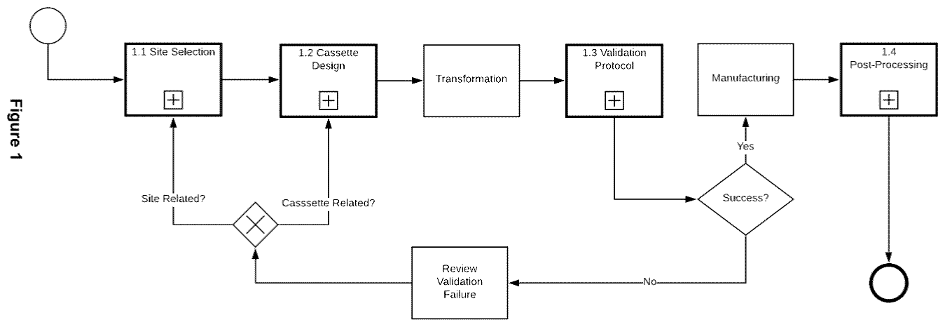

A summary flowchart of the method for inertly identifying a biological entity according to a preferred embodiment is depicted in FIG. 1.

### Selection of an Integration Site

The method first comprises receiving or providing a sample comprising the biological entity. The nucleic acid sequence of the biological entity instructs the selection of an appropriate integration site in the nucleic acid sequence for insertion of an inert cassette. Integration sites may have already been established for any given biological entity. As such, the selection step first entails determining if a new site is required using one or more databases well known the art. If a new site is required, the properties for a suitable site may be determined experimentally using a variety of molecular biology approaches well known in the art since these properties may vary from organism to organism, or strain to strain. After having determined the genetic landscape and considered a plurality of properties of the biological entity, a selection of a target site for insertion of an inert cassette is made within the constraints of the available genomic data.

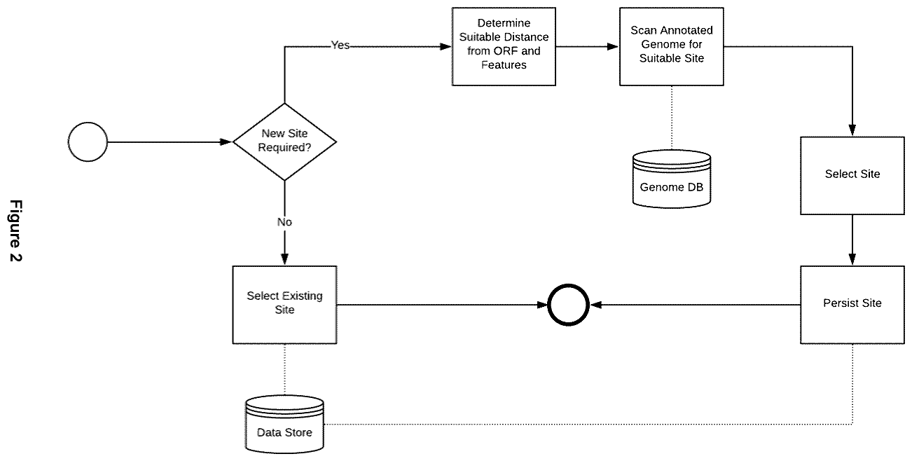

A summary flowchart of the method for selecting an appropriate integration site according to a preferred embodiment is depicted in FIG. 2.

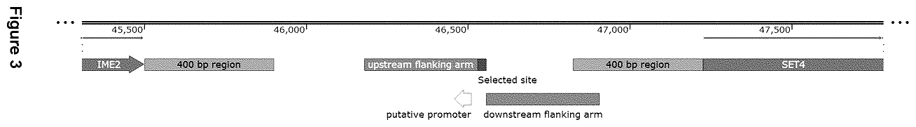

The selection of an integration site within Saccharomyces cerevisiae is depicted in FIG. 3. The selection first entails screening the host genome and identifying any characterized, putative genes, open reading frames and/or features of concern. Potential sites within a predetermined distance (measured in nucleotide base pairs, denoted bp) from characterized, putative genes, open reading frames and/or features of concern, in any direction on either nucleic acid strand are then selected. One criterion that may be used to identify potential sites is a distance of at least 250 bp since this distance is expected to be sufficient to avoid the disruption of the proximal promoter regions, which in eukaryotes are about 250 bp away from genes (Goni et al., 2007). The distance of at least 250 bp is also expected to be sufficient to avoid the disruption of the 5′ UTR in terminators, which typically ranges 100-200 bp away from stop codons across several life domains (Mignone et al., 2002). Another criterion which may be used to select the site is the absence of characterized and/or putative promoters directing to the transcription of the cassette, which may be screened in silico using, for example, methods described in Reese (2001). FIG. 3 illustrates a suitable site given the desired properties described hereinabove.

In addition, the selected sites may preferably be located within heterochromatic regions which are loci typically found near telomeres and centromeres that are characterized by tightly packed DNA. The position of heterochromatic genes in these regions is known to cause gene silencing (Gartenberg and Smith, 2016). It may be desired to integrate the cassette within heterochromatic regions in order to achieve silencing. Furthermore, the selection of sites may preferably be located within Long Terminal Repeats (LTRs) which are repeated regions within the genome that are reminiscent of a retroviral infection (Coffin et al., 1997). In addition to not disrupting critical genes native to the host organism, the integration of the cassette in LTR regions can have the added benefit of creating extra copies of the cassette, as these loci are often repeated in the genome dozens or even hundreds of times (Shi et al., 2016).

### Design of the Inert Cassette

Once the target site has been selected, the method follows with the design of an inert cassette having an arbitrary sequence for integration in the biological entity. In order to design an appropriate inert cassette without introducing or modifying characteristics in the biological entity, a stop codon configuration is selected from one or more host codon-optimized databases well known in the art. These stop codons may be variably distanced from each other and repeated throughout the cassette in pre-determined combinations. Once selected, the stop codon configuration is added to the cassette, which is then reviewed for any additional sequences of concern in view of the wild-type biological entity and databases of known nucleic acid motifs. The identified sequence(s) of concern is then disrupted until there are no remaining sequences of concern. In addition, any start codons that arise from the design will be disrupted. The resulting cassette's sequence is then stored in a database.

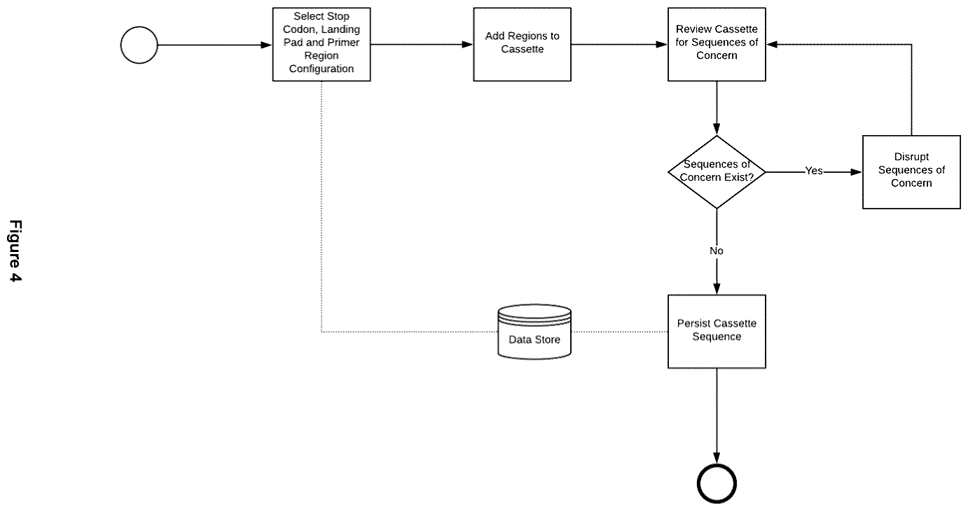

A summary flowchart of the method for designing the inert cassette, according to a preferred embodiment is depicted in FIG. 4.

An example of a cassette design is depicted in FIG. 5. The cassette may be up to ˜1500 bp long and may contain optimized primer sequences designed in silico, reproduced from the literature, or selected within the cassette for qPCR (Bustin and Huggett, 2017), sequencing (Dieffenbach et al., 1993), LAMP assays (Jia et al., 2019), RPA (Higgings et al., 2018), and/or other amplification techniques. The cassette may also contain sequences optimized in silico for qPCR probes (Bustin and Huggett, 2017), or sites for gRNA binding for use with CRISPR-Cas editing methods (Bourgeois et al., 2018). The cassette should contain stop codons for all of the 6 reading frames and in both the 3′ and 5′ ends of the cassette as well as on both sense and antisense strands, in order to stop any transcription initiation within the cassette and the sequences flanking the cassette. For large cassette designs that are, for example, larger than 100 bp, stop codon sequences for all reading frames will be randomly inserted in between every 50-300 bp, as depicted in FIG. 5. This stop codon design will mitigate risks associated with any previously undetected, low levels of transcribed RNA resulting in a functional protein product. A cut-off size of about 300 bp, or 100 amino acids, is typically the threshold for the annotation of open reading frames (ORFs) because peptides that are smaller than 100 amino acids are unlikely to have stability, folding capacity and/or biochemical activity (Dujon et al., 1994; White, 1994). For example, a 6 frame stop codon sequence may be TTAATTAATTAA (SEQ ID NO: 30). The cassette is designed such that start codons are removed or disrupted. In addition, the designed nucleic acid sequence of the inert cassette is screened in silico for absence of putative promoters (Reese, 2001) and other motifs of concern. If putative promoters or concerning motifs exist, the cassette is redesigned.

Integration of the cassette may be conducted with a CRISPR-Cas assisted double strand break followed by a homology-directed repair (DiCarlo et al., 2013). In this approach, the Cas nuclease is expressed in a species-specific high copy plasmid between a suitable species-specific promoter and terminator and selected by a dominant marker, for example, an antibiotic resistance gene. In addition, the plasmid contains a species-specific RNA polIII promoter to drive the expression of the gRNA compatible with the designated Cas, in which the spacer was selected to target the selected site. Other methods for integrating the inert nucleic acid cassette may be used while remaining within the scope of the present invention. The cassette may be produced as a synthetic nucleic acid block including homology arms of 75-500 bp that direct the nucleic acid repair and cassette integration within the site. A suitable nucleic acid transformation is performed and should be adapted for each organism. Thereafter, the transformed organisms are pre-screened for the presence of the nucleic acid sequence of the cassette at the desired site, a process sometimes called colony-PCR, a method that is described for example in Sheu et al., 2000. Other methods for prescreening for the presence of the nucleic acid sequence of the cassette at the desired site may be used while remaining within the scope of the present invention.

Once prescreen is completed, a positive organism is submitted to culturing in the absence of the plasmid selection, for example, in the absence of antibiotic, to remove the nuclease expression plasmid, a process that sometimes is called curing of the plasmid, as described by Rodríguez-López et al., 2017. After the strain is cured of nuclease plasmid, a series of validations can follow concurrently.

### Validation of the Inert Cassette

Once the cassette has been designed in silico, the cassette can then be synthesized experimentally and inserted into an appropriate plasmid using standard molecular biology techniques well known in the art or transformed on its own. The cassette-containing plasmid is then transformed into the biological entity for validation steps to ensure that the transformed biological entity meets the requirements for an inert bioengineering event. Alternatively, the cell is separately transfected with a gRNA-containing plasmid and the DNA cassette. The genomic nucleic acid of the biological entity is first extracted and submitted to whole genome sequencing (WGS), to assess if the cassette has the correct sequence, to confirm that it was integrated in the correct site, and to verify if any notable validation errors may have occurred. These validation errors include but are not limited to 1) Off-target effects review 2) Integration site review and 3) Cassette sequence review processes. If an off-target mutation event occurs, another colony can be screened. This can be repeated several times until a selected colony meets the validation criteria. If validation fails after a certain number of iterations, a redesign of the inert cassette may be required. It is not necessary that all of these validation steps be completed in each validation process run. The bioengineering validation steps are critical to ensure that the transformant meets the requirements for an inert bioengineering event. Additional validation steps may be taken or some validation steps may be omitted while remaining within the scope of the present invention. The complex gateway denoted by the [symbol] icons describes tasks within a process that may vary. However, where some set of tasks is required, that whole set must be completed successfully. As soon as a single task within that set fails, the whole process fails and terminates. In both cases (success and failure) the results are recorded in a database.

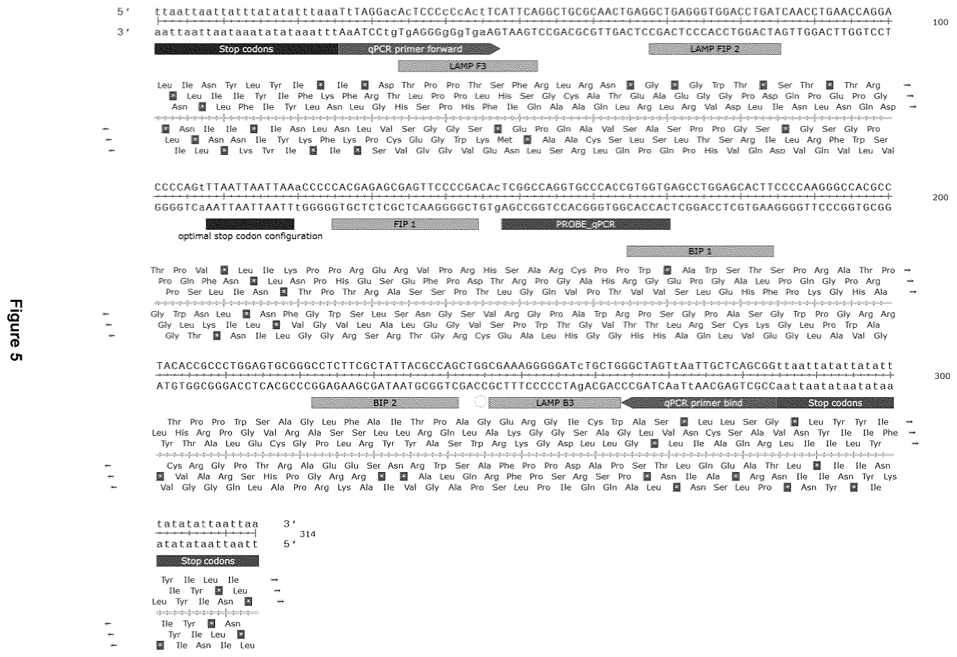

A summary flowchart of the method for validating that no characteristics have been added or modified in the biological entity, according to a preferred embodiment is depicted in FIG. 6.

In an exemplary embodiment, Saccharomyces cerevisiae S288C was transformed with an integration cassette of 370 bp in a site 810 bp distant of the gene TRM7 and 1461 bp distant from the gene OCR2 using CRISPR-Cas9 and gRNA expression plasmids with a hygromycin resistance gene. The prescreen colony-PCR was carried out by extracting the genomic DNA of 4 transformants and carrying out colony PCR with primers amplifying a region corresponding to 500 bp flanking the integration site in the wild-type strain. Agarose gel electrophoresis showed an increase in molecular weight of the DNA fragment corresponding to the inserted inert cassette. This indicated that the transformants had successfully integrated with the cassette in the selected site.

Validation of the genomic integration of the inert cassette into the biological entity is followed by a phenotypical characterization to validate that the integration of the cassette did not introduce any characteristics or modify the characteristics of the biological entity.

Accordingly, the biological entity is cultured under biomass production conditions and the RNA extracted for expression review. For example, expression review can be based on reverse transcriptase qPCR targeting the amplification of the cassette for quantifying the expression level of the cassette, (as described in Freeman et al., 1999) compared to the wild type strain without the cassette. If the signal is more than what is identified as the acceptable upper threshold, the site and/or the cassette may need to be redesigned. Other methods for expression review may be used while remaining within the scope of the present invention.

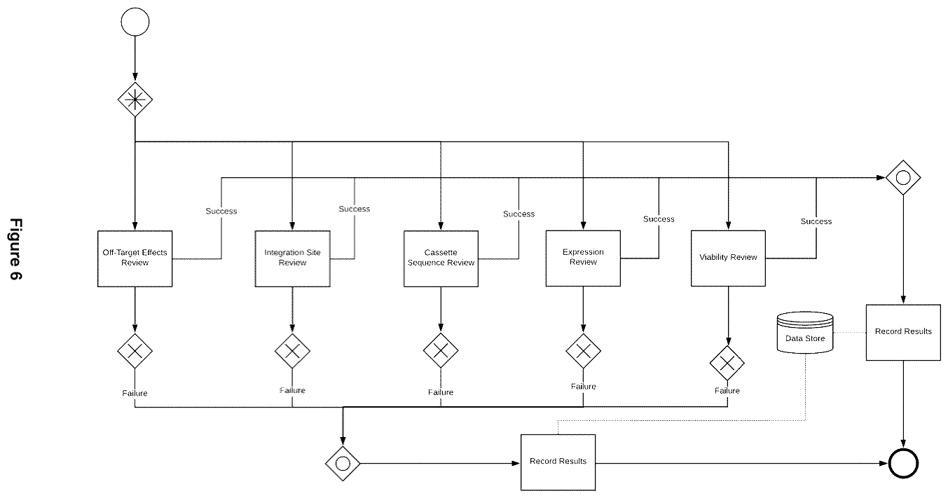

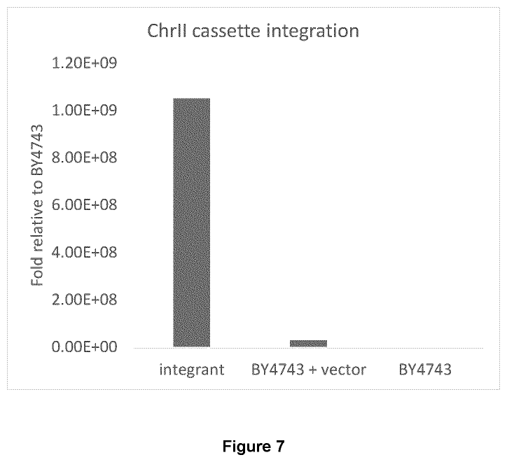

In the example depicted in FIG. 7, one of the pre-screened Saccharomyces cerevisiae S288C transformants was cultured in the biomass production conditions and its RNA was extracted (integrant), alongside with a strain where the same 370 bp cassette was cloned to a plasmid (BY4743+vector), and the wild type strain (BY4743). The RNA was reverse transcribed to cDNA, which then was submitted to qPCR using primers and probes that bind within the cassette. The integrant shows a significant fold increase in the expression of the cassette compared to what was observed from the cassette being incorporated in a plasmid (BY4743 +vector), or in the absence of the cassette (BY4743). This clearly shows that unforeseen RNA expression can occur depending on the combination of site/cassette DNA sequence, for example, in the insertion of the cassette downstream of an uncharacterized promoter.

The phenotypical characterization follows with a viability analysis. The biological entity comprising the inert cassette is cultured alongside and in the same conditions as the wild-type strain to assess if the growth curves overlap to determine whether the integration of the cassette perturbed any genes, metabolic regulation or any other features that might regulate growth.

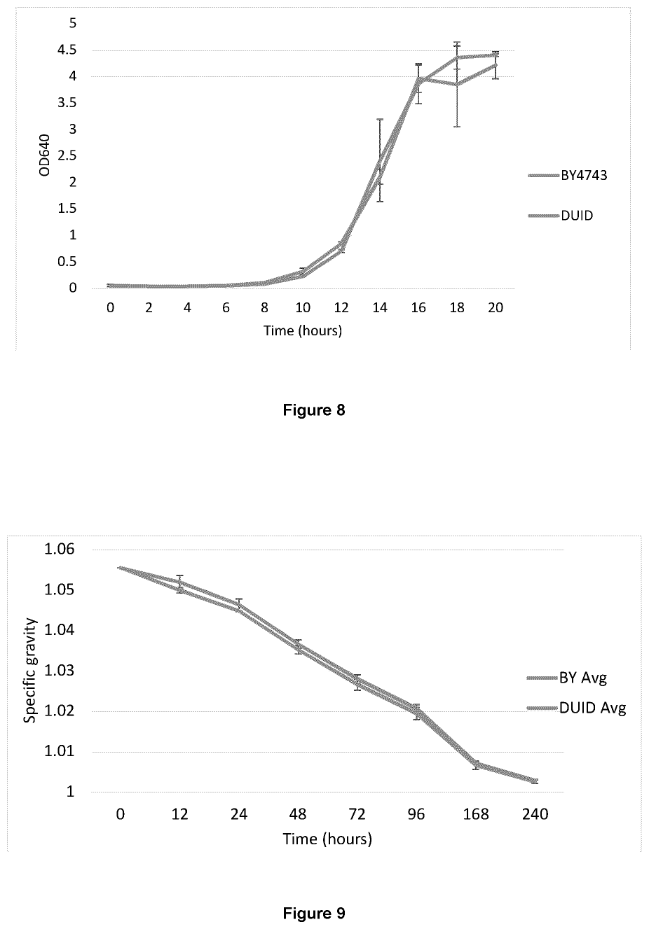

In the example depicted in FIG. 8, the growth curves of the wild type Saccharomyces cerevisiae S288C strain (green), and integrant (blue) overlap, as measured by the OD640 over time. This indicates that the introduction of the cassette did not introduce any characteristics that affect growth.

In addition, metabolic regulation may be assessed by comparing the sugar consumption profile of the biological entity containing the cassette to the wild type strain, as depicted in FIG. 9. In this example, the sugar consumption was followed using specific gravimetry (De Clerck, 1958). This can be carried out in microplates with automated plate reader or by manually taking samples overtime and accessing the change in the optical density signal, which correlates with biomass concentration, as described in Sonderegger and Sauer (2003). The curves of sugar consumption of the wild type Saccharomyces cerevisiae S288C strain (green), and integrant (blue) overlap, as measured by specific gravity. This indicates that the introduction of the cassette did not introduce any characteristics that affect sugar metabolism. It is to be understood that other biological characteristics well known the art may be evaluated to validate that no characteristics have been added or modified while still remaining within the scope of the present invention. For example, molecular and cellular signatures, sensitivity or response to the environment, reproduction, growth and development, regulation, homeostasis, and energy processing may be assessed.

The validated biological entity can then qualify for commercialization and stored in the strain repository. Alternatively, further validation steps may be undertaken to satisfy regulatory hurdles and/or to ensure that no characteristics have been modified or added to the biological entity. These further validation steps may include chemical equivalency and proteomics with mass spectrometry, for example.

### Post-Processing

According to a preferred embodiment, further post-processing steps are included to limit the potential for generational mutations and environmental release. Post-processing comprises inactivating the biological entity, followed by growth experiments which are well known in the art to ensure the inactivation was successful. The inactivation steps for a host organism is selected and adapted according to the selected species. A thermal inactivation (as in Couto et al., 2005), a chemical inactivation (Li and Wu, 2013), or a combination of these two processes may be carried out. For example, thermal inactivation can be accomplished by incubation of the fermentation broth containing the biomass, or biomass washed with water or buffer, at a certain temperature, for example 85° C., for a certain amount time, for example 20 minutes. Chemical inactivation can be accomplished by the addition of acetic acid to the fermentation broth containing the biomass, or biomass washed with water or buffer, to a certain concentration, for example, 5-1% v/v, letting it sit for a certain period of time in treatment, for example, 20 minutes. Cell inactivation can be followed by cell staining methods, such as trypan blue, which gives an indication of the extent of cellular death and as such indicates if the method was effective (Kucsera et al., 2000).

A summary flowchart of the method for inactivating the biological entity, according to a preferred embodiment is depicted in FIG. 10.

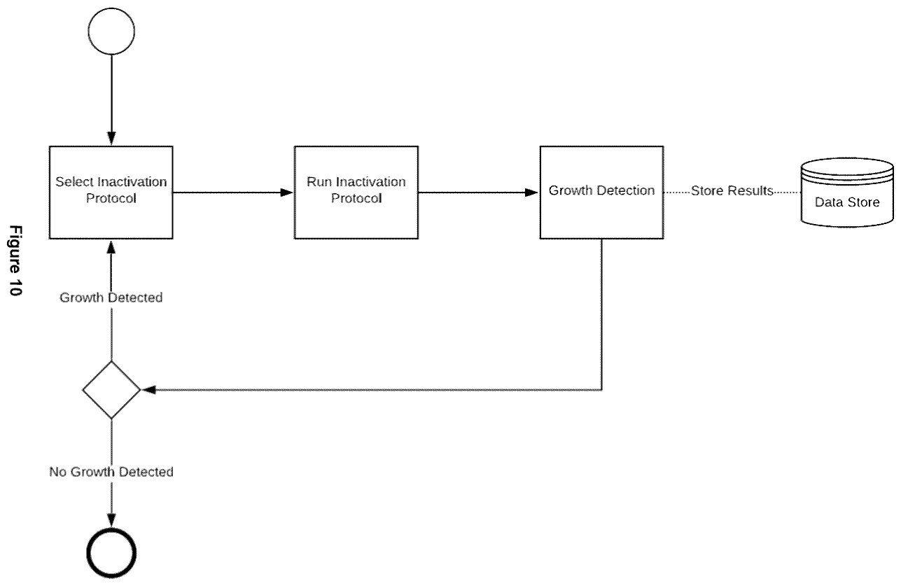

In the example depicted in FIG. 11, trypan blue staining was selected to assess cell death. The selected organism, Saccharomyces cerevisiae S288C, was incubated at 85° C. for 20 min. Cells stained with trypan blue and showing positive blue staining indicate cell death, which may in turn indicate that the treatment was effective, although further confirmation with CFU/mg would be required to make a definitive conclusion.

### Identification of Known Organisms

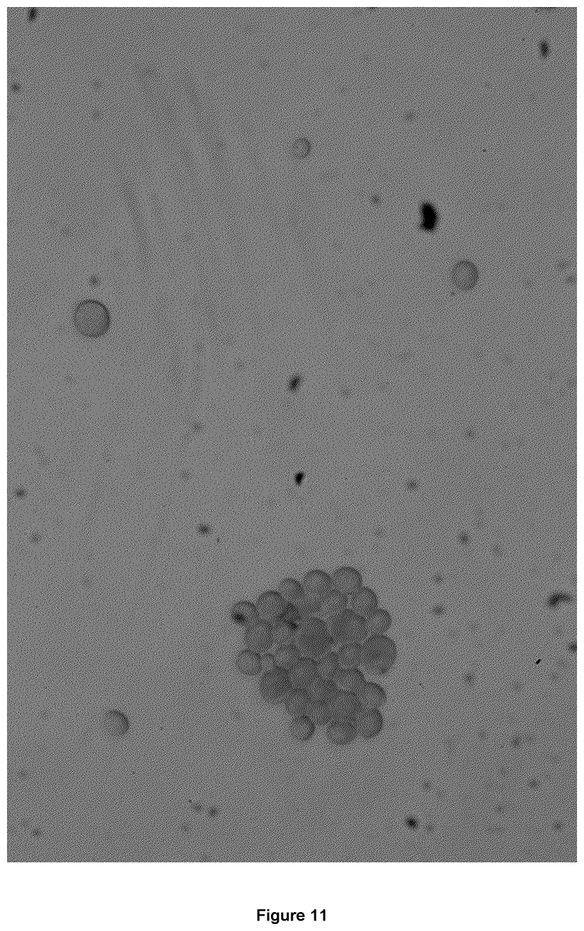

According to an embodiment, the design of an inert cassette may further comprise inserting an organism barcode sequence for identifying a known organism in a biological entity. The organism may be a known pathogen or a known allergen. The known organism to be identified and for which a barcode is inserted in the cassette may be selected according to pathogens which are known to contaminate any given biological entity. The pathogens may further be selected according to the epidemiological status of a known pathogen, for example, severe acute respiratory syndrome coronavirus 2 (SARS-COV-2) and the current global pandemic. Accordingly, current epidemiological status of the SARS-COV-2 would benefit from the identification of biological entities contaminated with SARS-COV-2 by allowing contaminated biological entities to be disinfected and/or destroyed, thereby mitigating the propagation of SARS-COV-2. The pathogen barcode sequence is designed from the known sequence of the known pathogen. An example of a cassette design comprising a pathogen barcode is depicted in FIG. 12. This particular pathogen barcode comprises a sequence from the SARS-COV-2, or 2019-nCOV, which can be identified using standard molecular biology techniques targeting the barcode. For example, PCR amplification of the barcode with a pair of primers targeting the sequence can yield an amplicon that can easily be detected using techniques that are well known in the art. The barcode can also be identified using a probe having a complementary sequence to a portion of the barcode wherein the probe can be detected by various techniques well known in the art. For example, the probe can comprise modified nucleotides (e.g., radioactive, fluorescent) which emit a detectable signal and/or the probe can be tagged with an antigen which can be detected with a specific antibody. Other means of detecting the barcode well known in the art may be used without deviating from the teachings of the present invention.

Preferably, the sequence of the barcode comprises a sequence that is commonly used in assays for the detection of that pathogen. This is because many pathogens cannot be released into the environment, which makes it almost impossible to test and validate many of the assays in real world circumstances. For example, African Swine Fever (ASF) cannot be studied in the U.S. and as such, this particular pathogen must be sent to England where the only labs allowed to study the virus are located. Although considerable progress has been made in the development of assays for the detection of ASF, there is need for positive controls to validate their efficacy, such as within final manufactured feed products, for which positive controls do not exist. Similarly, assays developed for the detection of many other pathogens within the context of air quality monitoring efforts also lack appropriate positive controls for validating the assays.

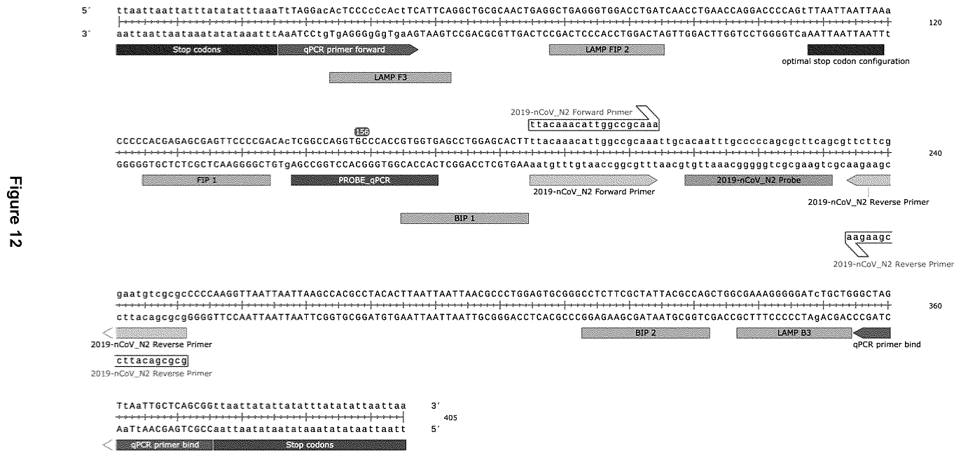

Another example of a cassette design with a pathogen barcode comprising a sequence from SARS-COV-2 (2019-nCOV) is depicted in FIG. 13. This particular pathogen barcode comprises randomized sequences flanking the probe sequence and nested within the primers. The randomized sequences provide additional differentiation from the pathogen sequence without affecting general properties of the inert cassette (length, probe and primers). Indeed, these randomized sequences for providing additional differentiation further ensure that the bioengineering event is an inert bioengineering event. These additional differentiation sequences can therefore address potential concerns and/or regulatory hurdles regarding the inclusion of pathogenic DNA in organisms used within the supply chain products and materials. These concerns are especially relevant when the amplicon size is larger, for example over 500 bp.

It is to be understood that the addition of a barcode to the cassette can be also be used to identify any organism having a known sequence. For example, the method disclosed herein can also be used for the detection of known allergens or any other organisms for which detection within supply chain products and materials would be desirable. According to an embodiment, multiple DNA barcodes from several known organisms may be inserted into the cassette to allow the identification of multiple organisms in a biological entity from a single cassette. As such, the inert bioengineering event can provide a single product having multiple sequences capable of identifying multiple organisms. The single product can serve as a microorganism-based positive control library for validating the efficacy of current assays. Various other products may be developed without deviating from the teachings of the present invention. For example, in order to ensure that the bioengineering event is an inert bioengineering event, different products may be developed having sequences from different organisms in order for the cassette to remain an acceptable size to qualify as an inert bioengineering event. The acceptable size will vary depending on the organism and context of the insertion site. It is to be understood that various libraries comprising different categories of organisms may also be developed while remaining within the scope of the invention.

### EXAMPLES

### Inert Cassette Design & Validation

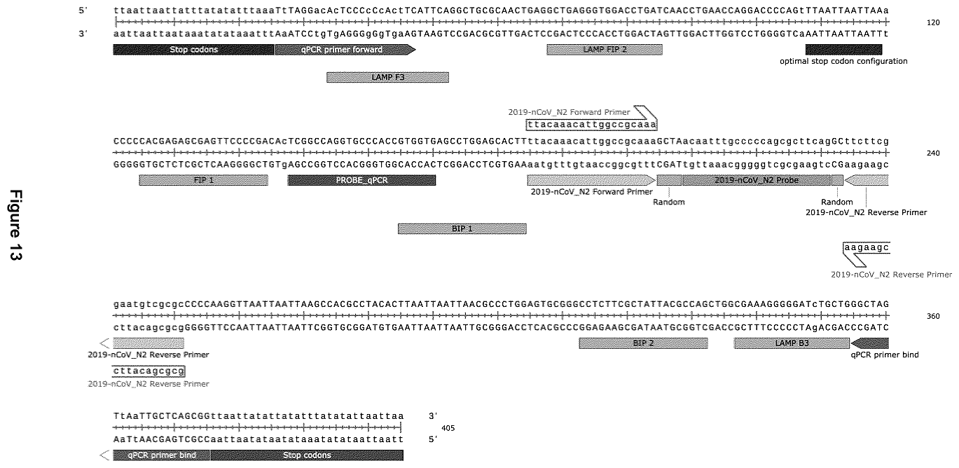

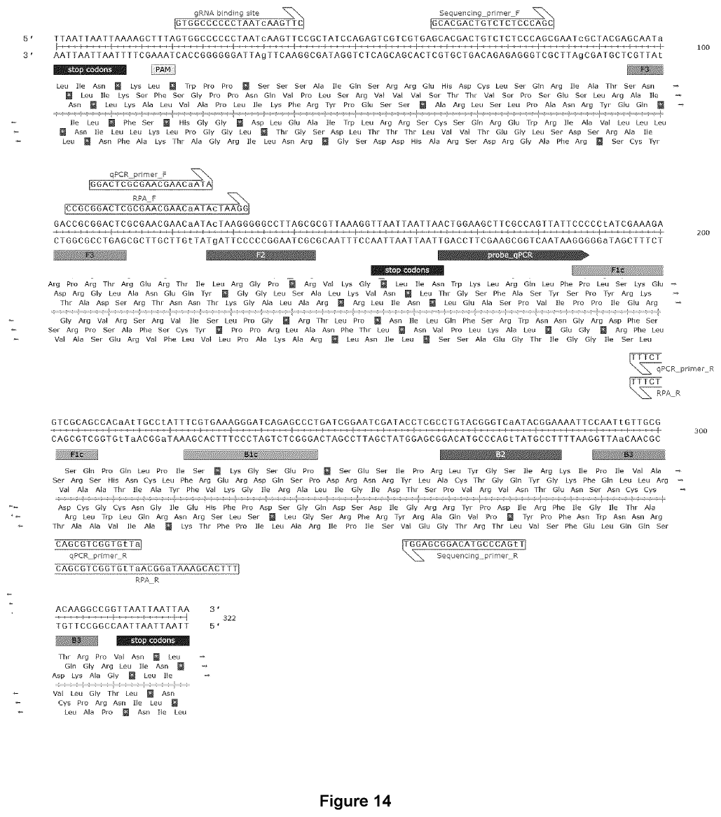

Another example of an inert cassette design is depicted in FIG. 14 (SEQ ID NO: 29). The cassette is 322 bp long and contains optimized primers designed in silico for sequencing, qPCR, loop mediated isothermal amplification (LAMP), and recombinase polymerase amplification (RPA). The cassette also contains optimized qPCR probe and gRNA binding sequences. The cassette also contains strategically distributed (i.e. optimized) stop codons for all 6 reading frames (TTAATTAATTAA; SEQ ID NO: 30). These stop codons are in both flanks of the cassette as well as within the cassette. The inert nucleic acid cassette was screened for putative promoter sequences using the method described in Reese (2001).

FIG. 15 (SEQ ID NO: 135) illustrates a selected site for cassette integration, located in the intergenic region of two galactose utilization genes GAL10 and GAL7 at chromosome II of Saccharomyces cerevisiae. The selected site is positioned 291 bp downstream of the gene GAL7 (SEQ ID NO: 56) and 428 bp upstream of the gene GAL10 (SEQ ID NO: 58). The inert nucleic acid cassette described in FIG. 14 (SEQ ID NO: 29), was integrated in the site noted in FIG. 15. Integration was confirmed with Sanger sequencing and WGS.

FIG. 16 (SEQ ID NO: 138) illustrates a selected site for cassette integration, located in the intergenic region of the genes MTR2 and ASHI at chromosome XI of Saccharomyces cerevisiae. The selected site is positioned 737 bp downstream of the gene MTR2 (SEQ ID NO:

60) and 463 bp upstream of the gene ASHI (SEQ ID NO: 63). The inert cassette described in FIG. 14, was integrated in the site noted in FIG. 16. Integration was confirmed with Sanger sequencing and WGS.

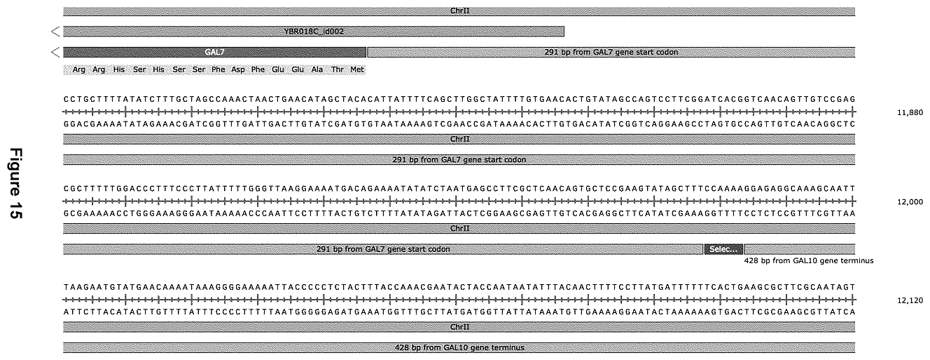

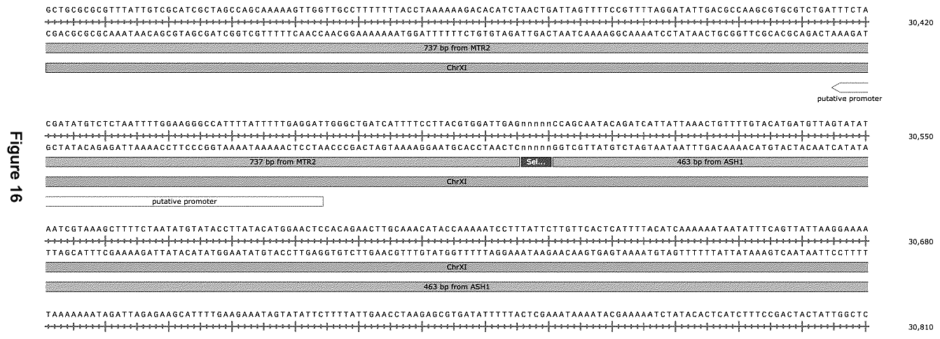

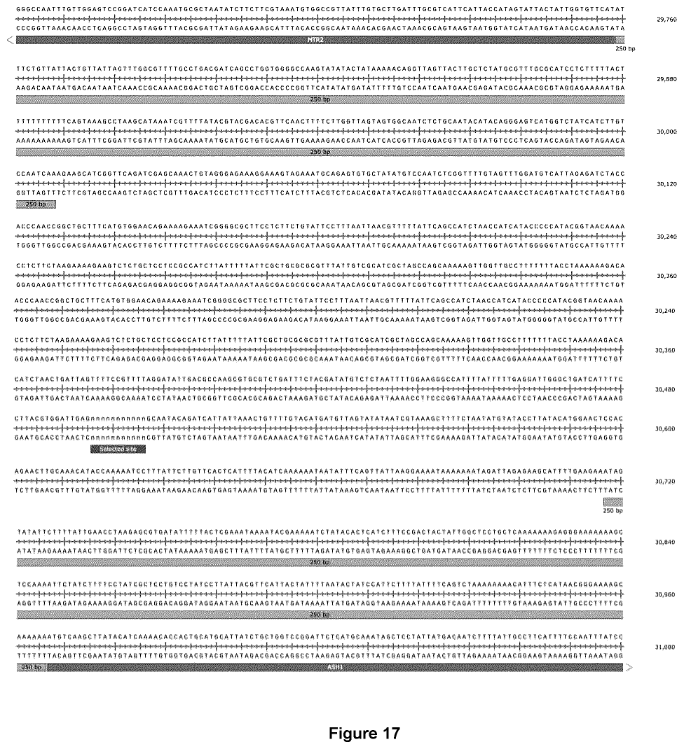

FIG. 17 (SEQ ID NO: 140) illustrates a selected site located in the intergenic region of the genes IME2 and SET4 at chromosome X of Saccharomyces cerevisiae. The selected site is positioned 1045 bp downstream of the gene IME2 and 682 bp upstream of the gene SET4. The inert cassette described in FIG. 14, was integrated in the site noted in FIG. 17. Integration was confirmed with Sanger sequencing and WGS.

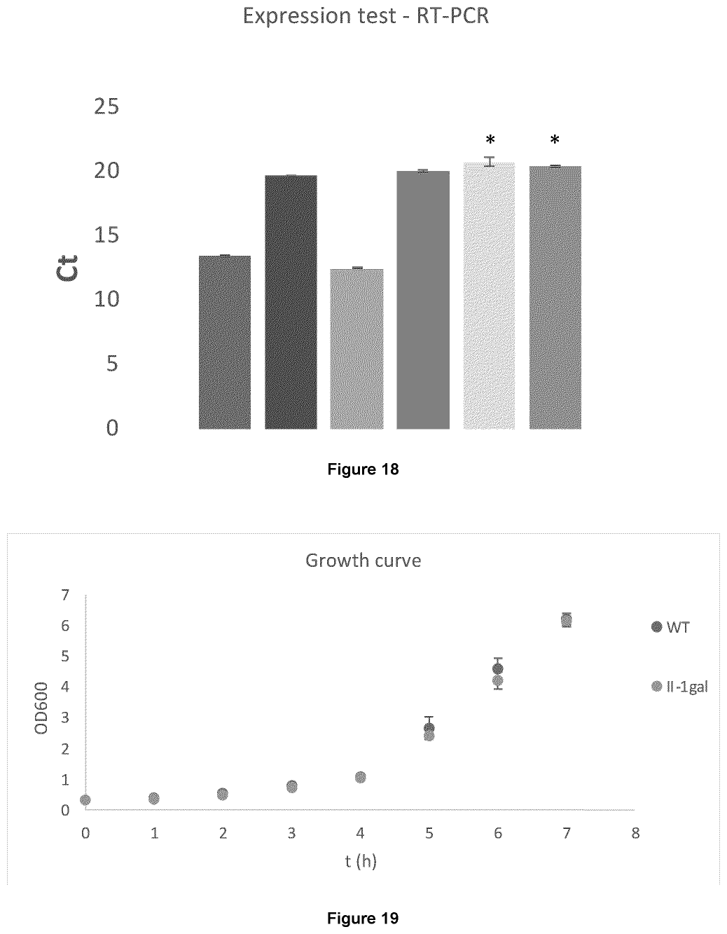

In the example depicted in FIG. 18, a Saccharomyces cerevisiae strain engineered with the inert cassette illustrated in FIG. 14 at the selected site depicted in FIG. 15, was cultured in the biomass production conditions and its RNA was extracted. The RNA was reverse transcribed to cDNA, which then was analyzed via qPCR using primers and probes that bind within the cassette. The integrant shows no statistically significant increase in the expression of the cassette (cDNA; 5th bar from the left) compared to the no-RT control (NRT; 6th bar from the left). Internal controls were used for the genes ACTI (cDNA; 1st bar from the left, NRT; 2nd bar from the left) and FBA (cDNA; 3rd bar from the left, NRT; 4th bar from the left). This shows RNA expression was not detectable for this engineering event.

FIG. 19 illustrates the growth curves of a wild type Saccharomyces cerevisiae strain (blue) compared to the Saccharomyces cerevisiae strain engineered with the inert cassette illustrated in FIG. 14 at the selected site depicted in FIG. 15 (Selec . . . ). In this case, the curves overlap, as measured by the OD600 over time. This indicates that the introduction of the cassette did not introduce any characteristics that affect growth with statistical significance.

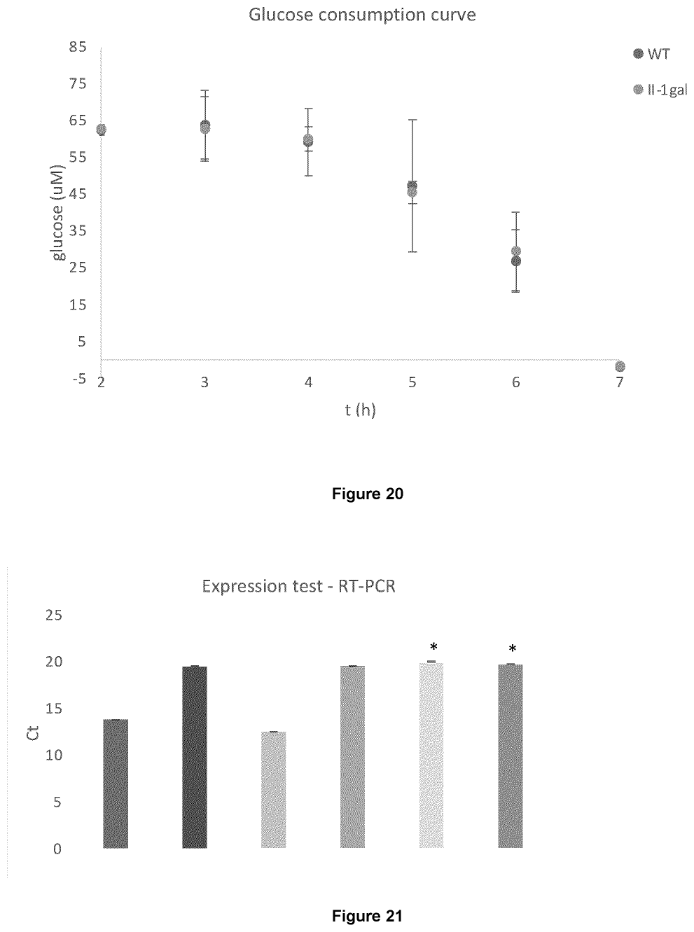

In the example depicted in FIG. 20, the sugar consumption curves of the wild type Saccharomyces cerevisiae strain (blue), is compared to the Saccharomyces cerevisiae strain engineered with the inert cassette illustrated in FIG. 14 at the selected site depicted in FIG. 15 (Selec . . . ). In this case, the curves of sugar consumption overlap, as measured by Amplex red glucose assay. This indicates that the introduction of the cassette did not introduce any characteristics that affect sugar metabolism with statistical significance.

In the example depicted in FIG. 21, a Saccharomyces cerevisiae strain engineered with the inert cassette illustrated in FIG. 14 at the selected site depicted in FIG. 16, was cultured in the biomass production conditions and its RNA was extracted. The RNA was reverse transcribed to cDNA, which then was submitted to qPCR using primers and probes that bind within the cassette. The integrant shows no significant increase in the expression of the cassette (cDNA; 5th bar from the left) compared to the no-RT control (NRT; 6th bar from the left). Internal controls were used for the genes ACTI (cDNA; 1st bar from the left, NRT; 2nd bar from the left) and FBA (cDNA; 3rd bar from the left, NRT; 4th bar from the left). This shows RNA expression was not detectable for this engineering event.

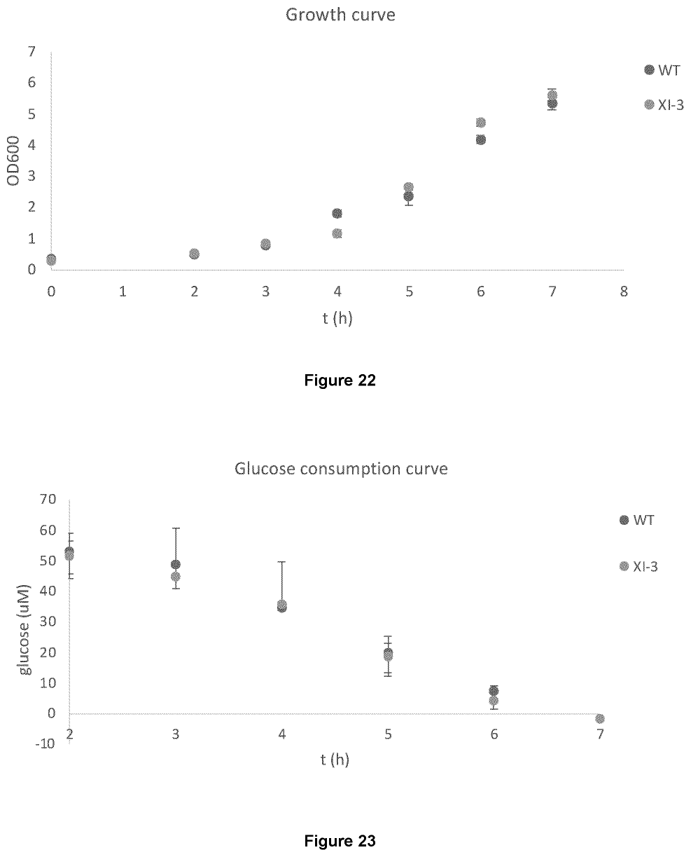

In the example depicted in FIG. 22, the growth curves of the wild type Saccharomyces cerevisiae strain (blue), are compared to a Saccharomyces cerevisiae strain engineered with the inert cassette illustrated in FIG. 14 at the selected site depicted in FIG. 16 (Selec . . . ). In this case, the curves overlap, as measured by the OD600 over time. This indicates that the introduction of the cassette did not introduce any characteristics that affect growth with statistical significance.

In the example depicted in FIG. 23, the sugar consumption curves of the wild type Saccharomyces cerevisiae strain (WT-top circle at 2, 3, 5 and 6 hours), and a Saccharomyces cerevisiae strain engineered with the inert cassette described in FIG. 14, at the selected site depicted in FIG. 16 (Selec . . . ). In this case, the curves of sugar consumption overlap, as measured by Amplex red glucose assay. This indicates that the introduction of the cassette did not introduce any characteristics that affect sugar metabolism with statistical significance.

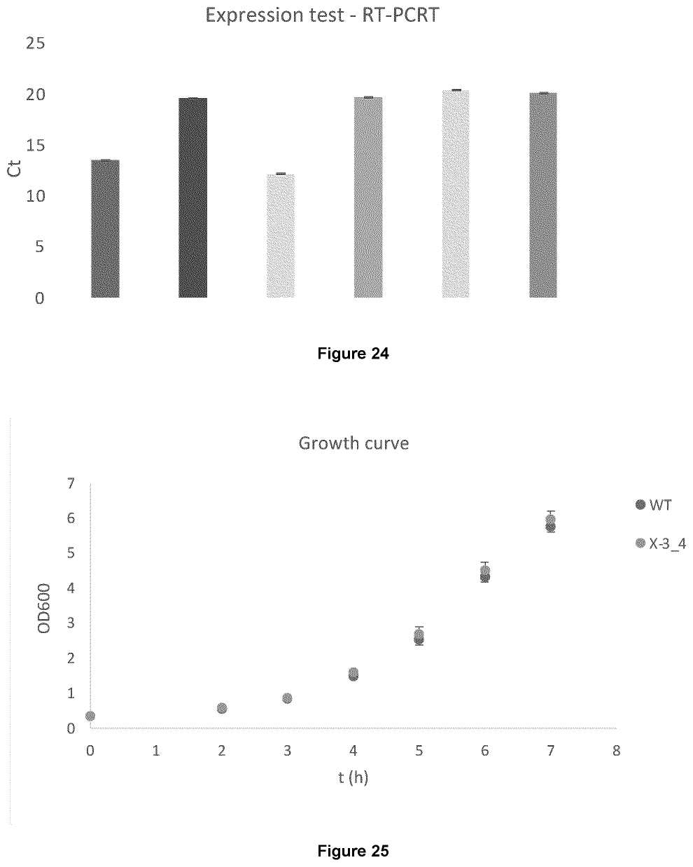

In the example depicted in FIG. 24, a Saccharomyces cerevisiae strain engineered with the inert cassette illustrated in FIG. 14, at the selected site depicted in FIG. 17 (Selected site), was cultured in the biomass production conditions and its RNA was extracted. The RNA was reverse transcribed to cDNA, which then was analyzed via qPCR using primers and probes that bind within the cassette. The integrant shows no statistically significant increase in the expression of the cassette (cDNA; 5th bar from the left) compared to the no-RT control (NRT; 6th bar from the left). Internal controls were used for the genes ACTI (cDNA; 1st bar from the left, NRT; 2nd bar from the left) and FBA (cDNA; 3rd bar from the left, NRT; 4th bar from the left). This shows RNA expression was not detectable for this engineering event.

In the example depicted in FIG. 25, the growth curves of the wild type Saccharomyces cerevisiae strain (blue), and the Saccharomyces cerevisiae strain engineered with the inert cassette illustrated in FIG. 14 at the selected site depicted in FIG. 17 (Selected site) are shown. In this case, the curves overlap, as measured by the OD600 over time. This indicates that the introduction of the cassette did not introduce any characteristics that affect growth with statistical significance.

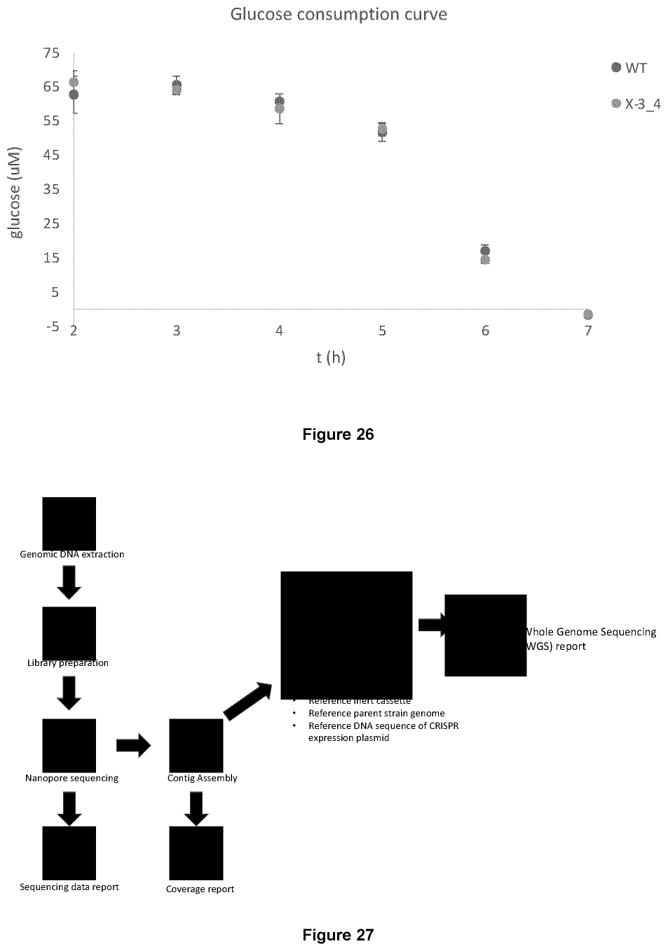

In the example depicted in FIG. 26, the sugar consumption curves of the wild type Saccharomyces cerevisiae strain (blue), and a Saccharomyces cerevisiae strain engineered with the inert cassette illustrated in FIG. 14, at the selected site depicted in FIG. 17 (Selected site) are shown. In this case, the curves of sugar consumption overlap, as measured by Amplex red glucose assay. This indicates that the introduction of the cassette did not introduce any characteristics that affect sugar metabolism with statistical significance.

In the example depicted in FIG. 27, WGS was carried out using nanopore sequencing technique, and the raw reads were validated through a data quality report. The raw reads were assembled into contigs representing yeast's chromosomes, with adequate coverage confirmed. Next, the assembled genome was aligned with the reference sequence of the inert cassette, generating a visual representation of the aligned data. Additionally, the assembled contig was aligned against the reference genome of the parental strain to ensure no additional sequence insertions have occurred. From the alignment, it was concluded that the complete sequence of the inert cassette was integrated into the correct locus, without additional integrations in unintended loci. The assembled genome was also searched for sequences of the CRISPR-Cas9 and gRNA expression plasmid, and no elements of the plasmid was found unintentionally integrated. Thus, these WGS results were able to validate that no other genomic characteristics were added or modified, thereby demonstrating the suitability of the method of the present invention for bioengineering a biological entity without introducing or modifying characteristics in the biological entity.

While illustrative and presently preferred embodiments of the invention have been described in detail hereinabove, it is to be understood that the inventive concepts may be otherwise variously embodied and employed and that the appended claims are intended to be construed to include such variations except insofar as limited by the prior art.

### REFERENCES

Bourgeois, L., Pyne, M. E., & Martin, V. J. J. (2018). A Highly Characterized Synthetic Landing Pad System for Precise Multicopy Gene Integration in Yeast. ACS Synthetic Biology, 7(11), 2675-2685.
Bustin, S., & Huggett, J. (2017). qPCR primer design revisited. Biomolecular Detection and Quantification, 14, 19-28.
Coffin J M, Hughes S H, Varmus H E, editors. Cold Spring Harbor (NY): Cold Spring Harbor Laboratory Press; 1997.
Couto, J. A., Neves, F., Campos, F., & Hogg, T. (2005). Thermal inactivation of the wine spoilage yeasts Dekkera/Brettanomyces. International Journal of Food Microbiology, 104(3), 337-344.
De Clerck, J. A textbook of Brewing, Translated by Kathleen Barton-Wright, Chapman & Hall,, London 1958 Vol. II p427.
DiCarlo, J. E., Norville, J. E., Mali, P., Rios, X., Aach, J., & Church, G. M. (2013). Genome engineering in Saccharomyces cerevisiae using CRISPR-Cas systems. Nucleic Acids Research, 41(7), 4336-4343.
Dieffenbach, C. W., Lowe, T. M., & Dveksler, G. S. (1993). General concepts for PCR primer design. Genome Research, 3(3), S30-S37.
Dujon B, Alexandraki D, André B, Ansorge W, Baladron V, Ballesta J P G, Banrevi A, Bolle P A, Bolotin-Fukuhara M, Bossier P, et al. Complete DNA sequence of yeast chromosome XI. Nature. 1994;369:371.
Freeman, W. M., Walker, S. J., & Vrana, K. E. (1999). Quantitative RT-PCR: Pitfalls and Potential. BioTechniques, 26(1), 112-125.
Gartenberg, M. R., & Smith, J. S. (2016). The Nuts and Bolts of Transcriptionally Silent Chromatin in Saccharomyces cerevisiae. Genetics, 203(4), 1563-1599.
Goñi, J. R., Pérez, A., Torrents, D., & Orozco, M. (2007). Determining promoter location based on DNA structure first-principles calculations. Genome Biology, 8(12), R263.
Higgins, M., Ravenhall, M., Ward, D., Phelan, J., Ibrahim, A., Forrest, M. S., Clark, T. G., & Campino, S. (2018). PrimedRPA: primer design for recombinase polymerase amplification assays. Bioinformatics, 35(4), 682-684.
Jia, B., Li, X., Liu, W., Lu, C., Lu, X., Ma, L., Li, Y.-Y., & Wei, C. (2019). GLAPD: Whole Genome Based LAMP Primer Design for a Set of Target Genomes. Frontiers in Microbiology, 10.
Kucsera, J., Yarita, K., & Takeo, K. (2000). Simple detection method for distinguishing dead and living yeast colonies. Journal of Microbiological Methods, 41(1), 19-21.
Li, Y., & Wu, C. (2013). Enhanced inactivation of Salmonella Typhimurium from blueberries by combinations of sodium dodecyl sulfate with organic acids or hydrogen peroxide. Food Research International, 54(2), 1553-1559.
Mignone, F., Gissi, C., Liuni, S., & Pesole, G. (2002). Genome Biology, 3(3), reviews0004.1 .
Reese, M. G. (2001). Application of a time-delay neural network to promoter annotation in the Drosophila melanogaster genome. Computers & Chemistry, 26(1), 51-56.
Rodríguez-López, M., Cotobal, C., Fernández-Sánchez, O., Borbarán Bravo, N., Oktriani, R., Abendroth, H., Uka, D., Hoti, M., Wang, J., Zaratiegui, M., & Bähler, J. (2017). A CRISPR/Cas9-based method and primer design tool for seamless genome editing in fission yeast. Wellcome Open Research, 1, 19.
Sheu, D.-S., Wang, Y.-T., & Lee, C.-Y. (2000). Rapid detection of polyhydroxyalkanoate-accumulating bacteria isolated from the environment by colony PCR. Microbiology, 146(8), 2019-2025.
Shi, S., Liang, Y., Zhang, M. M., Ang, E. L., & Zhao, H. (2016). A highly efficient single-step, markerless strategy for multi-copy chromosomal integration of large biochemical pathways in Saccharomyces cerevisiae. Metabolic Engineering, 33, 19-27.
Sonderegger, M., & Sauer, U. (2003). Evolutionary Engineering of Saccharomyces cerevisiae for Anaerobic Growth on Xylose. Applied and Environmental Microbiology, 69(4), 1990-1998.
White, S. H. (1994). The evolution of proteins from random amino acid sequences: II. Evidence from the statistical distributions of the lengths of modern protein sequences. Journal of Molecular Evolution, 38(4), 383-394.

### EMBODIMENTS

1. A method for bioengineering a biological entity by introducing an inert nucleic acid cassette without introducing or modifying characteristics in the biological entity, the method comprising:
receiving or providing a sample comprising the biological entity having a nucleic acid sequence;
selecting an integration site in the nucleic acid sequence for inserting the inert nucleic acid cassette;
designing the inert cassette with optimized primer sequences, optimized probe sequences, optimized stop codons and disrupted start codons, and inserting the inert nucleic acid cassette into the biological entity at the integration site; and
validating that no characteristics have been added or modified in the biological entity.

2. The method according to claim 1, wherein selecting an integration site comprises determining if a new site is required.

3. The method according to claim 2, wherein determining if a new site is required comprises consulting at least one database to verify if a prior engineering event has occurred in the biological entity.

4. The method according to claim 1, wherein selecting an integration site comprises screening the genome of the biological entity and identifying any characterized putative genes, open reading frames and/or features of concern.

5. The method according to claim 4, wherein the features of concern comprises putative promoters, enhancers and any other known regulatory sequences.

6. The method according to claim 1, wherein the selected integration site is within a predetermined distance in base pairs (bp) from known or putative genes, open reading frames and/or features of concern.

7. The method according to claim 6, wherein the predetermined distance is between about 250 bp and about 1 kilobase pairs (kb).

8. The method according to claim 7, wherein the predetermined distance is about 250 bp.

9. The method according to claim 1, wherein the selected integration site is located within heterochromatic regions and/or long terminal repeats (LTRs).

10. The method according to claim 1, wherein selecting an integration site comprises determining an absence of characterized and/or putative promoters directing transcription of the cassette.

11. The method according to claim 1, wherein designing the inert cassette comprises creating landing pads for assisting with the integration of the inert cassette at the integration site.

12. The method according to claim 11, wherein the landing pad is a CRISPR target sequence complementary to an optimized gRNA spacer.

13. The method according to claim 1, wherein the integration site is selected in silico using at least one database.

14. The method according to claim 1, further comprising inactivating the biological entity.

15. The method according to claim 14, wherein inactivating the biological entity comprises thermal inactivation, chemical inactivation, nutrient deprivation over time or combinations thereof.

16. The method according to claim 1, wherein validating that no characteristics have been added or modified in the biological entity comprises analyzing the transcriptional profile of the biological entity to validate that transcripts of the inert nucleic acid cassette cannot be detected above a predetermined threshold.

17. The method according to claim 1, wherein validating that no characteristics have been added or modified in the biological entity comprises sequencing the whole genome of the biological entity to validate that the inert nucleic acid cassette has been integrated at the selected site, without any off-target effects.

18. The method according to claim 17, wherein the off-target effects comprises mutations in the cassette or elsewhere in the genome of the biological entity caused by the bioengineering process.

19. The method according to claim 1, wherein designing the inert cassette further comprises inserting at least one barcode sequence from at least one organism having a known sequence for detecting the at least one organism in the biological entity.

20. The method according to claim 19, wherein the at least one organism is a pathogen and/or an allergen.

21. The method according to claim 19, wherein the at least one barcode sequence comprises randomized sequences for differentiating the barcode sequence from the sequence of the at least one organism.

22. An inert nucleic acid cassette for identifying a biological material, the inert nucleic acid cassette comprising optimized primer sequences, optimized probe sequences, optimized stop codons and disrupted start codons.

23. The inert nucleic acid cassette according to claim 22, wherein the optimized stop codons comprises stop codons for all reading frames in order to stop any transcription initiation within the cassette and the sequences flanking the cassette.

24. The inert nucleic acid cassette according to claim 22 or 23, further comprising at least one barcode sequence from at least one organism having a known sequence for detecting the at least one organism in the biological entity.

25. The inert nucleic acid cassette according to claim 24, wherein the at least one organism is a pathogen and/or an allergen.

26. The inert nucleic acid cassette according to claim 24 or 25, further comprising randomized sequences for differentiating the at least one barcode sequence from the sequence of the at least one organism.

1. A method for bioengineering a biological entity by introducing an inert nucleic acid cassette without introducing or modifying characteristics in the biological entity, the method comprising:
receiving or providing a sample comprising the biological entity having a nucleic acid sequence;
selecting an integration site in the nucleic acid sequence for inserting the inert nucleic acid cassette;
designing the inert cassette with optimized primer sequences, optimized probe sequences, optimized stop codons and disrupted start codons, and inserting the inert nucleic acid cassette into the biological entity at the integration site; and
validating that no characteristics have been added or modified in the biological entity.

2. The method according to claim 1, wherein selecting an integration site comprises:
i) determining if a new site is required, wherein determining if a new site is required comprises consulting at least one database to verify if a prior engineering event has occurred in the biological entity;
ii) screening the genome of the biological entity and identifying any characterized putative genes, open reading frames and/or features of concern, wherein the features of concern comprises putative promoters, enhancers and any other known regulatory sequences;
iii) determining an absence of characterized and/or putative promoters directing transcription of the cassette; or
iv) any combination thereof.

3.-5. (canceled)

6. The method according to claim 1, wherein the selected integration site is within a predetermined distance in base pairs (bp) from putative genes, open reading frames and/or features of concern.

7. The method according to claim 6, wherein the predetermined distance is between about 250 bp and about 1 kilobase pairs (kb).

8. (canceled)

9. The method according to claim 1, wherein the selected integration site is located within heterochromatic regions and/or long terminal repeats (LTRs).

10. (canceled)

11. The method according to claim 1, wherein designing the inert cassette comprises creating landing pads for assisting with the integration of the inert cassette at the integration site.

12. The method according to claim 11, wherein the landing pad is a CRISPR target sequence complementary to an optimized gRNA spacer.

13.-14. (canceled)

15. The method according to claim 1, wherein inserting the inert nucleic acid cassette into the biological entity at the integration site is performed using homology arms of between about 75 to about 500 bp for directing nucleic acid and integration of the inert nucleic acid cassette within the site.

16. The method according to claim 1, further comprising inactivating the biological entity.

17. (canceled)

18. The method according to claim 1, wherein validating that no characteristics have been added or modified in the biological entity comprises:
i) analyzing the transcriptional profile of the biological entity to validate that transcripts of the inert nucleic acid cassette cannot be detected above a predetermined threshold;
ii) sequencing the whole genome of the biological entity to validate that the inert nucleic acid cassette has been integrated at the selected site, without any off-target effects, wherein the off-target effects comprise mutations in the cassette or elsewhere in the genome of the biological entity;
iii) evaluating sensitivity and/or responses of the biological entity to a variety of environmental stimuli;
iv) evaluating growth and development;
v) evaluating homeostasis;
vi) evaluating metabolic regulation, wherein evaluating metabolic regulation is accomplished by measuring sugar consumption and/or oxygen consumption;
vii) evaluating function of known regulatory pathways;
viii) a chemical equivalency assessment and/or proteomics analysis; or
ix) any combination thereof.

19.-23. (canceled)

24. The method according to claim 1, wherein designing the inert cassette further comprises inserting at least one barcode sequence from at least one organism having a known sequence for detecting the at least one organism in the biological entity.

25. The method according to claim 24, wherein the at least one organism is a pathogen and/or an allergen.

26. The method according to claim 25, wherein the pathogen is a virus, bacterium, protozoan, prion, viroid, or fungus.

27.-28. (canceled)

29. The method according to claim 24, wherein the at least one barcode sequence comprises randomized sequences for differentiating the barcode sequence from the sequence of the at least one organism.

30. An inert nucleic acid cassette for identifying a biological material, the inert nucleic acid cassette comprising optimized primer sequences, optimized probe sequences, optimized stop codons and disrupted start codons.

31. The inert nucleic acid cassette according to claim 30, wherein the optimized stop codons comprises optimized stop codons for all reading frames in order to stop any transcription initiation within the cassette and the sequences flanking the cassette.

32. The inert nucleic acid cassette according to claim 31, wherein the inert nucleic acid cassette comprises optimized stop codons for all reading frames at regular intervals.

33. (canceled)

34. The inert nucleic acid cassette according to claim 32, wherein the sequence of the optimized stop codons for all reading frames is TTAATTAATTAA (SEQ ID NO: 30).

35. The inert nucleic acid cassette according to claim 30, further comprising at least one barcode sequence from at least one organism having a known sequence for detecting the at least one organism in the biological entity.

36.-39. (canceled)

40. The inert nucleic acid cassette according to claim 35, further comprising randomized sequences for differentiating the at least one barcode sequence from the sequence of the at least one organism.


SEQUENCE LISTING (141 sequences)

SEQ ID NO: 1-10 - skipped (10 entries)

SEQ ID NO: 11 - DNA, 314 bp, synthetic construct
  source 1..314: mol_type = other DNA
ttaattaattatttatatatttaaatttaggacactccccccacttcattcaggctgcgcaactgaggctgagggtggacctgatcaacctgaaccaggaccccagtttaattaattaaacccccacgagagcgagttccccgacactcggccaggtgcccaccgtggtgagcctggagcacttccccaagggccacgcctacaccgccctggagtgcgggcctcttcgctattacgccagctggcgaaagggggatctgctgggctagttaattgctcagcggttaattatattatatttatatattaattaa

SEQ ID NO: 12 - DNA, 25 bp, synthetic construct
  source 1..25: mol_type = other DNA
ttaattaattatttatatatttaaa

SEQ ID NO: 13 - DNA, 22 bp, synthetic construct
  source 1..22: mol_type = other DNA
tttaggacactccccccacttc

SEQ ID NO: 14 - DNA, 19 bp, synthetic construct
  source 1..19: mol_type = other DNA
actccccccacttcattca

SEQ ID NO: 15 - DNA, 18 bp, synthetic construct
  source 1..18: mol_type = other DNA
gctgagggtggacctgat

SEQ ID NO: 16 - DNA, 20 bp, synthetic construct
  source 1..20: mol_type = other DNA
cacgagagcgagttccccga

SEQ ID NO: 17 - DNA, 23 bp, synthetic construct
  source 1..23: mol_type = other DNA
tcggccaggtgcccaccgtggtg

SEQ ID NO: 18 - DNA, 20 bp, synthetic construct
  source 1..20: mol_type = other DNA
gtggtgagcctggagcactt

SEQ ID NO: 19 - DNA, 20 bp, synthetic construct
  source 1..20: mol_type = other DNA
cctcttcgctattacgccag

SEQ ID NO: 20 - DNA, 18 bp, synthetic construct
  source 1..18: mol_type = other DNA
cgaaagggggatctgctg

SEQ ID NO: 21 - DNA, 21 bp, synthetic construct
  source 1..21: mol_type = other DNA
ccgctgagcaattaactagcc

SEQ ID NO: 22 - DNA, 30 bp, synthetic construct
  source 1..30: mol_type = other DNA
ttaattatattatatttatatattaattaa

SEQ ID NO: 23-28 - skipped (6 entries)

SEQ ID NO: 29 - DNA, 322 bp, synthetic construct
  source 1..322: mol_type = other DNA
ttaattaattaaaagctttagtggcccccctaatcaagttccgctatccagagtcgtcgtgagcacgactgtctctcccagcgaatcgctacgagcaatagaccgcggactcgcgaacgaacaatactaagggggccttagcgcgttaaaggttaattaattaactggaagcttcgccagttattccccctatcgaaagagtcgcagccacaattgcctatttcgtgaaagggatcagagccctgatcggaatcgatacctcgcctgtacgggtcaatacggaaaattccaattgttgcgacaaggccggttaattaattaa

SEQ ID NO: 30 - DNA, 12 bp, synthetic construct
  source 1..12: mol_type = other DNA
ttaattaattaa

SEQ ID NO: 31 - DNA, 21 bp, synthetic construct
  source 1..21: mol_type = other DNA
gtggcccccctaatcaagttc

SEQ ID NO: 32 - DNA, 20 bp, synthetic construct
  source 1..20: mol_type = other DNA
gcacgactgtctctcccagc

SEQ ID NO: 33 - DNA, 18 bp, synthetic construct
  source 1..18: mol_type = other DNA
gcaatagaccgcggactc

SEQ ID NO: 34 - DNA, 30 bp, synthetic construct
  source 1..30: mol_type = other DNA
ccgcggactcgcgaacgaacaatactaagg

SEQ ID NO: 35 - DNA, 20 bp, synthetic construct
  source 1..20: mol_type = other DNA
ggactcgcgaacgaacaata

SEQ ID NO: 36 - DNA, 18 bp, synthetic construct
  source 1..18: mol_type = other DNA
actaagggggccttagcg

SEQ ID NO: 37 - skipped

SEQ ID NO: 38 - DNA, 25 bp, synthetic construct
  source 1..25: mol_type = other DNA
actggaagcttcgccagttattccc

SEQ ID NO: 39 - DNA, 22 bp, synthetic construct
  source 1..22: mol_type = other DNA
ccccctatcgaaagagtcgcag

SEQ ID NO: 40 - DNA, 30 bp, synthetic construct
  source 1..30: mol_type = other DNA
tttcacgaaataggcaattgtggctgcgac

SEQ ID NO: 41 - DNA, 19 bp, synthetic construct
  source 1..19: mol_type = other DNA
attgtggctgcgactcttt

SEQ ID NO: 42 - DNA, 22 bp, synthetic construct
  source 1..22: mol_type = other DNA
ttcgtgaaagggatcagagccc

SEQ ID NO: 43 - DNA, 20 bp, synthetic construct
  source 1..20: mol_type = other DNA
ttgacccgtacaggcgaggt

SEQ ID NO: 44 - DNA, 20 bp, synthetic construct
  source 1..20: mol_type = other DNA
cctgtacgggtcaatacgga

SEQ ID NO: 45 - DNA, 19 bp, synthetic construct
  source 1..19: mol_type = other DNA
ccaattgttgcgacaaggc

SEQ ID NO: 46 - skipped

SEQ ID NO: 47 - protein, 107 aa, synthetic construct
  VARIANT 4: note = Xaa can be any naturally occuring amino acid
  VARIANT 7: note = Xaa can be any naturally occuring amino acid
  VARIANT 11: note = Xaa can be any naturally occuring amino acid
  VARIANT 47: note = Xaa can be any naturally occuring amino acid
  VARIANT 52: note = Xaa can be any naturally occuring amino acid
  VARIANT 76: note = Xaa can be any naturally occuring amino acid
  VARIANT 82: note = Xaa can be any naturally occuring amino acid
  VARIANT 106: note = Xaa can be any naturally occuring amino acid
LINXKLXWPPXSSSAIQSRREHDCLSQRIATSNRPRTRERTILRGPXRVKGXLINWKLRQLFPLSKESQPQLPISXKGSEPXSESIPRLYGSIRKIPIVATRPVNXL

SEQ ID NO: 48 - protein, 107 aa, synthetic construct
  VARIANT 1: note = Xaa can be any naturally occuring amino acid
  VARIANT 43: note = Xaa can be any naturally occuring amino acid
  VARIANT 53: note = Xaa can be any naturally occuring amino acid
  VARIANT 107: note = Xaa can be any naturally occuring amino acid
XLIKSFSGPPNQVPLSRVVVSTTVSPSESLRAIDRGLANEQYXGGLSALKVNXLTGSFASYSPYRKSRSHNCLFRERDQSPDRNRYLACTGQYGKFQLLRQGRLINX

SEQ ID NO: 49 - protein, 106 aa, synthetic construct
  VARIANT 2: note = Xaa can be any naturally occuring amino acid
  VARIANT 20: note = Xaa can be any naturally occuring amino acid
  VARIANT 33: note = Xaa can be any naturally occuring amino acid
  VARIANT 49: note = Xaa can be any naturally occuring amino acid
  VARIANT 54: note = Xaa can be any naturally occuring amino acid
  VARIANT 104: note = Xaa can be any naturally occuring amino acid
NXLKALVAPLIKFRYPESSXARLSLPANRYEQXTADSRTNNTKGALARXRLINXLEASPVIPPIERVAATIAYFVKGIRALIGIDTSPVRVNTENSNCCDKAGXLI

SEQ ID NO: 50 - protein, 107 aa, synthetic construct
  VARIANT 4: note = Xaa can be any naturally occuring amino acid
  VARIANT 35: note = Xaa can be any naturally occuring amino acid
  VARIANT 54: note = Xaa can be any naturally occuring amino acid
  VARIANT 59: note = Xaa can be any naturally occuring amino acid
  VARIANT 78: note = Xaa can be any naturally occuring amino acid
  VARIANT 93: note = Xaa can be any naturally occuring amino acid
  VARIANT 106: note = Xaa can be any naturally occuring amino acid
LINXPALSQQLEFSVLTRTGEVSIPIRALIPFTKXAIVAATLSIGGITGEASSXLINLXRAKAPLVLFVRESAVYCSXRFAGRDSRAHDDSGXRNLIRGATKAFNXL

SEQ ID NO: 51 - protein, 107 aa, synthetic construct
  VARIANT 1: note = Xaa can be any naturally occuring amino acid
  VARIANT 16: note = Xaa can be any naturally occuring amino acid
  VARIANT 29: note = Xaa can be any naturally occuring amino acid
  VARIANT 44: note = Xaa can be any naturally occuring amino acid
  VARIANT 47: note = Xaa can be any naturally occuring amino acid
  VARIANT 55: note = Xaa can be any naturally occuring amino acid
  VARIANT 65: note = Xaa can be any naturally occuring amino acid
  VARIANT 96: note = Xaa can be any naturally occuring amino acid
  VARIANT 107: note = Xaa can be any naturally occuring amino acid
XLINRPCRNNWNFPYXPVQARYRFRSGLXSLSRNRQLWLRLFRXGEXLAKLPVNXLTFNALRPPXYCSFASPRSIARSDSLGETVVLTTTLDSGTXLGGPLKLLINX

SEQ ID NO: 52 - protein, 105 aa, synthetic construct
  VARIANT 2: note = Xaa can be any naturally occuring amino acid
  VARIANT 55: note = Xaa can be any naturally occuring amino acid
  VARIANT 60: note = Xaa can be any naturally occuring amino acid
  VARIANT 96: note = Xaa can be any naturally occuring amino acid
  VARIANT 100: note = Xaa can be any naturally occuring amino acid
  VARIANT 103: note = Xaa can be any naturally occuring amino acid
NXLTGLVATIGIRIDPYRRGIDSDQGSDPFHEIGNCGCDSFDRGNNWRSFQLINXPLTRXGPLSIVRSRVRGLLLVAIRWERQSCSRRLWIAELDXGGHXSFXLI

SEQ ID NO: 53-55 - skipped (3 entries)

SEQ ID NO: 56 - DNA, 291 bp, synthetic construct
  source 1..291: mol_type = other DNA
ttttgagggaatattcaactgtttttttttatcatgttgatgctctgcataataatgcccataaatatttccgacctgcttttatatctttgctagccaaactaactgaacatagctacacattattttcagcttggctattttgtgaacactgtatagccagtccttcggatcacggtcaacagttgtccgagcgctttttggaccctttcccttatttttgggttaaggaaaatgacagaaaatatatctaatgagccttcgctcaacagtgctccgaagtatagcttt

SEQ ID NO: 57 - skipped

SEQ ID NO: 58 - DNA, 137 bp, synthetic construct
  source 1..137: mol_type = other DNA
ggagaggcaaagcaatttaagaatgtatgaacaaaataaaggggaaaaattaccccctctactttaccaaacgaatactaccaataatatttacaacttttccttatgattttttcactgaagcgcttcgcaatagt

SEQ ID NO: 59 - skipped

SEQ ID NO: 60 - DNA, 177 bp, synthetic construct
  source 1..177: mol_type = other DNA
gccagcaaaaagttggttgcctttttttacctaaaaaagacacatctaactgattagttttccgttttaggatattgacgccaagcgtgcgtctgatttctacgatatgtctctaattttggaagggccattttatttttgaggattgggctgatcattttccttacgtggattgag

SEQ ID NO: 61 - DNA, 50 bp, synthetic construct
  source 1..50: mol_type = other DNA
tttctacgatatgtctctaattttggaagggccattttatttttgaggat

SEQ ID NO: 62 - DNA, 1524 bp, synthetic construct
  variation 15..1514: note = n is a, c, g, or t
  source 1..1524: mol_type = other DNA
ttacgtggattgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccagcaatac

SEQ ID NO: 63 - DNA, 310 bp, synthetic construct
  source 1..310: mol_type = other DNA
ccagcaatacagatcattattaaactgttttgtacatgatgttagtatataatcgtaaagcttttctaatatgtataccttatacatggaactccacagaacttgcaaacataccaaaaatcctttattcttgttcactcattttacatcaaaaaataatatttcagttattaaggaaaataaaaaaatagattagagaagcattttgaagaaatagtatattcttttattgaacctaagagcgtgatatttttactcgaaataaaatacgaaaaatctatacactcatctttccgactactattggctc

SEQ ID NO: 64-70 - skipped (7 entries)

SEQ ID NO: 71 - DNA, 405 bp, synthetic construct
  source 1..405: mol_type = other DNA
aattaattaataaatatataaatttaaatcctgtgaggggggtgaagtaagtccgacgcgttgactccgactcccacctggactagttggacttggtcctggggtcaaattaattaatttgggggtgctctcgctcaaggggctgtgagccggtccacgggtggcaccactcggacctcgtgaaaatgtttgtaaccggcgtttaacgtgttaaacgggggtcgcgaagtcgcaagaagccttacagcgcgggggttccaattaattaattcggtgcggatgtgaattaattaattgcgggacctcacgcccggagaagcgataatgcggtcgaccgctttccccctagacgacccgatcaattaacgagtcgccaattaatataatataaatatataattaatt

SEQ ID NO: 72 - protein, 7 aa, synthetic construct
LINYLYI

SEQ ID NO: 73 - protein, 11 aa, synthetic construct
DTPPTSFRLRN

SEQ ID NO: 74 - protein, 5 aa, synthetic construct
TRTPV

SEQ ID NO: 75 - protein, 19 aa, synthetic construct
LIKPPRERVPRHSARCPPW

SEQ ID NO: 76 - protein, 33 aa, synthetic construct
AWSTSPRATPTPPWSAGLFAITPAGERGICWAS

SEQ ID NO: 77 - protein, 4 aa, synthetic construct
LLSG

SEQ ID NO: 78 - protein, 8 aa, synthetic construct
LYYIYILI

SEQ ID NO: 79 - protein, 36 aa, synthetic construct
LIIYIFKFRTLPPLHSGCATEAEGGPDQPEPGPQFN

SEQ ID NO: 80 - protein, 64 aa, synthetic construct
LNPHESEFPDTRPGAHRGEPGALPQGPRLHRPGVRASSLLRQLAKGGSAGLVNCSAVNYIIFIY

SEQ ID NO: 81 - protein, 36 aa, synthetic construct
LFIYLNLGHSPHFIQAAQLRLRVDLINLNGDPSLIN

SEQ ID NO: 82 - protein, 49 aa, synthetic construct
TPTRASSPTLGQVPTVVSLEHFPKGHAYTALECGPLRYYASWRKGDLLG

SEQ ID NO: 83 - protein, 14 aa, synthetic construct
LIAQRLIILYLYIN

SEQ ID NO: 84 - protein, 4 aa, synthetic construct
LINI

SEQ ID NO: 85 - protein, 4 aa, synthetic construct
PLSN

SEQ ID NO: 86 - protein, 57 aa, synthetic construct
LAQQIPLSPAGVIAKRPALQGGVGVALGEVLGEVLQAHHGGHLAECRGTRSRGGLIN

SEQ ID NO: 87 - protein, 26 aa, synthetic construct
TGVLVQVDQVHPQPQLRSLNEVGGVS

SEQ ID NO: 88 - protein, 10 aa, synthetic construct
LIYKYNIINR

SEQ ID NO: 89 - protein, 10 aa, synthetic construct
PSRSPFRQLA

SEQ ID NO: 90 - protein, 9 aa, synthetic construct
RRGPHSRAV

SEQ ID NO: 91 - protein, 27 aa, synthetic construct
AWPLGKCSRLTTVGTWPSVGELALVGV

SEQ ID NO: 92 - protein, 21 aa, synthetic construct
LIKLGSWFRLIRSTLSLSCAA

SEQ ID NO: 93 - protein, 14 aa, synthetic construct
MKWGECPKFKYINN

SEQ ID NO: 94 - protein, 5 aa, synthetic construct
YINII

SEQ ID NO: 95 - protein, 58 aa, synthetic construct
LTAEQLTSPADPPFASWRNSEEARTPGRCRRGPWGSAPGSPRWAPGRVSGNSLSWGFN

SEQ ID NO: 96 - protein, 8 aa, synthetic construct
LNWGPGSG

SEQ ID NO: 97 - protein, 12 aa, synthetic construct
SGPPSASVAQPE

SEQ ID NO: 98 - protein, 10 aa, synthetic construct
SGGSVLNLNI

SEQ ID NO: 99 - DNA, 405 bp, synthetic construct
  source 1..405: mol_type = other DNA
ttaattaattatttatatatttaaatttaggacactccccccacttcattcaggctgcgcaactgaggctgagggtggacctgatcaacctgaaccaggaccccagtttaattaattaaacccccacgagagcgagttccccgacactcggccaggtgcccaccgtggtgagcctggagcacttttacaaacattggccgcaaattgcacaatttgcccccagcgcttcagcgttcttcggaatgtcgcgcccccaaggttaattaattaagccacgcctacacttaattaattaacgccctggagtgcgggcctcttcgctattacgccagctggcgaaagggggatctgctgggctagttaattgctcagcggttaattatattatatttatatattaattaa

SEQ ID NO: 100 - DNA, 405 bp, synthetic construct
  source 1..405: mol_type = other DNA
ttaattaatatataaatataatataattaaccgctgagcaattaactagcccagcagatccccctttcgccagctggcgtaatagcgaagaggcccgcactccagggcgttaattaattaagtgtaggcgtggcttaattaattaaccttgggggcgcgacattccgaagaacgctgaagcgctgggggcaaattgtgcaatttgcggccaatgtttgtaaaagtgctccaggctcaccacggtgggcacctggccgagtgtcggggaactcgctctcgtgggggtttaattaattaaactggggtcctggttcaggttgatcaggtccaccctcagcctcagttgcgcagcctgaatgaagtggggggagtgtcctaaatttaaatatataaataattaattaa

SEQ ID NO: 101 - DNA, 20 bp, synthetic construct
  source 1..20: mol_type = other DNA
ttacaaacattggccgcaaa

SEQ ID NO: 102 - DNA, 18 bp, synthetic construct
  source 1..18: mol_type = other DNA
gcgcgacattccgaagaa

SEQ ID NO: 103 - DNA, 405 bp, synthetic construct
  source 1..405: mol_type = other DNA
ttaattaattatttatatatttaaatttaggacactccccccacttcattcaggctgcgcaactgaggctgagggtggacctgatcaacctgaaccaggaccccagtttaattaattaaacccccacgagagcgagttccccgacactcggccaggtgcccaccgtggtgagcctggagcacttttacaaacattggccgcaaagctaacaatttgcccccagcgcttcaggcttcttcggaatgtcgcgcccccaaggttaattaattaagccacgcctacacttaattaattaacgccctggagtgcgggcctcttcgctattacgccagctggcgaaagggggatctgctgggctagttaattgctcagcggttaattatattatatttatatattaattaa

SEQ ID NO: 104 - DNA, 405 bp, synthetic construct
  source 1..405: mol_type = other DNA
ttaattaatatataaatataatataattaaccgctgagcaattaactagcccagcagatccccctttcgccagctggcgtaatagcgaagaggcccgcactccagggcgttaattaattaagtgtaggcgtggcttaattaattaaccttgggggcgcgacattccgaagaagcctgaagcgctgggggcaaattgttagctttgcggccaatgtttgtaaaagtgctccaggctcaccacggtgggcacctggccgagtgtcggggaactcgctctcgtgggggtttaattaattaaactggggtcctggttcaggttgatcaggtccaccctcagcctcagttgcgcagcctgaatgaagtggggggagtgtcctaaatttaaatatataaataattaattaa

SEQ ID NO: 105 - DNA, 322 bp, synthetic construct
  source 1..322: mol_type = other DNA
ttaattaattaaccggccttgtcgcaacaattggaattttccgtattgacccgtacaggcgaggtatcgattccgatcagggctctgatccctttcacgaaataggcaattgtggctgcgactctttcgatagggggaataactggcgaagcttccagttaattaattaacctttaacgcgctaaggcccccttagtattgttcgttcgcgagtccgcggtctattgctcgtagcgattcgctgggagagacagtcgtgctcacgacgactctggatagcggaacttgattaggggggccactaaagcttttaattaattaa

SEQ ID NO: 106 - protein, 35 aa, synthetic construct
SSSAIQSRREHDCLSQRIATSNRPRTRERTILRGP

SEQ ID NO: 107 - protein, 4 aa, synthetic construct
RVKG

SEQ ID NO: 108 - protein, 23 aa, synthetic construct
LINWKLRQLFPLSKESQPQLPIS

SEQ ID NO: 109 - protein, 5 aa, synthetic construct
KGSEP

SEQ ID NO: 110 - protein, 23 aa, synthetic construct
SESIPRLYGSIRKIPIVATRPVN

SEQ ID NO: 111 - protein, 41 aa, synthetic construct
LIKSFSGPPNQVPLSRVVVSTTVSPSESLRAIDRGLANEQY

SEQ ID NO: 112 - protein, 9 aa, synthetic construct
GGLSALKVN

SEQ ID NO: 113 - protein, 53 aa, synthetic construct
LTGSFASYSPYRKSRSHNCLFRERDQSPDRNRYLACTGQYGKFQLLRQGRLIN

SEQ ID NO: 114 - protein, 17 aa, synthetic construct
LKALVAPLIKFRYPESS

SEQ ID NO: 115 - protein, 12 aa, synthetic construct
ARLSLPANRYEQ

SEQ ID NO: 116 - protein, 15 aa, synthetic construct
TADSRTNNTKGALAR

SEQ ID NO: 117 - protein, 4 aa, synthetic construct
RLIN

SEQ ID NO: 118 - protein, 49 aa, synthetic construct
LEASPVIPPIERVAATIAYFVKGIRALIGIDTSPVRVNTENSNCCDKAG

SEQ ID NO: 119 - protein, 30 aa, synthetic construct
PALSQQLEFSVLTRTGEVSIPIRALIPFTK

SEQ ID NO: 120 - protein, 18 aa, synthetic construct
AIVAATLSIGGITGEASS

SEQ ID NO: 121 - protein, 4 aa, synthetic construct
LINL

SEQ ID NO: 122 - protein, 18 aa, synthetic construct
RAKAPLVLFVRESAVYCS

SEQ ID NO: 123 - protein, 14 aa, synthetic construct
RFAGRDSRAHDDSG

SEQ ID NO: 124 - protein, 12 aa, synthetic construct
RNLIRGATKAFN

SEQ ID NO: 125 - protein, 14 aa, synthetic construct
LINRPCRNNWNFPY

SEQ ID NO: 126 - protein, 12 aa, synthetic construct
PVQARYRFRSGL

SEQ ID NO: 127 - protein, 14 aa, synthetic construct
SLSRNRQLWLRLFR

SEQ ID NO: 128 - protein, 7 aa, synthetic construct
LAKLPVN

SEQ ID NO: 129 - protein, 9 aa, synthetic construct
LTFNALRPP

SEQ ID NO: 130 - protein, 30 aa, synthetic construct
YCSFASPRSIARSDSLGETVVLTTTLDSGT

SEQ ID NO: 131 - protein, 10 aa, synthetic construct
LGGPLKLLIN

SEQ ID NO: 132 - protein, 52 aa, synthetic construct
LTGLVATIGIRIDPYRRGIDSDQGSDPFHEIGNCGCDSFDRGNNWRSFQLIN

SEQ ID NO: 133 - protein, 4 aa, synthetic construct
PLTR

SEQ ID NO: 134 - protein, 35 aa, synthetic construct
GPLSIVRSRVRGLLLVAIRWERQSCSRRLWIAELD

SEQ ID NO: 135 - DNA, 360 bp, synthetic construct
  source 1..360: mol_type = other DNA
cctgcttttatatctttgctagccaaactaactgaacatagctacacattattttcagcttggctattttgtgaacactgtatagccagtccttcggatcacggtcaacagttgtccgagcgctttttggaccctttcccttatttttgggttaaggaaaatgacagaaaatatatctaatgagccttcgctcaacagtgctccgaagtatagctttccaaaaggagaggcaaagcaatttaagaatgtatgaacaaaataaaggggaaaaattaccccctctactttaccaaacgaatactaccaataatatttacaacttttccttatgattttttcactgaagcgcttcgcaatagt

SEQ ID NO: 136 - DNA, 360 bp, synthetic construct
  source 1..360: mol_type = other DNA
actattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcagg

SEQ ID NO: 137 - protein, 15 aa, synthetic construct
RRHSHSSFDFEEATM

SEQ ID NO: 138 - DNA, 517 bp, synthetic construct
  variation 206..210: note = n is a, c, g, or t
  source 1..517: mol_type = other DNA
gctgcgcgcgtttattgtcgcatcgctagccagcaaaaagttggttgcctttttttacctaaaaaagacacatctaactgattagttttccgttttaggatattgacgccaagcgtgcgtctgatttctacgatatgtctctaattttggaagggccattttatttttgaggattgggctgatcattttccttacgtggattgagnnnnngcaatacagatcattattaaactgttttgtacatgatgttagtatataatcgtaaagcttttctaatatgtataccttatacatggaactccacagaacttgcaaacataccaaaaatcctttattcttgttcactcattttacatcaaaaaataatatttcagttattaaggaaaataaaaaaatagattagagaagcattttgaagaaatagtatattcttttattgaacctaagagcgtgatatttttactcgaaataaaatacgaaaaatctatacactcatctttccgactactattggctc

SEQ ID NO: 139 - DNA, 517 bp, synthetic construct
  variation 308..312: note = n is a, c, g, or t
  source 1..517: mol_type = other DNA
gagccaatagtagtcggaaagatgagtgtatagatttttcgtattttatttcgagtaaaaatatcacgctcttaggttcaataaaagaatatactatttcttcaaaatgcttctctaatctatttttttattttccttaataactgaaatattattttttgatgtaaaatgagtgaacaagaataaaggatttttggtatgtttgcaagttctgtggagttccatgtataaggtatacatattagaaaagctttacgattatatactaacatcatgtacaaaacagtttaataatgatctgtattgcnnnnnctcaatccacgtaaggaaaatgatcagcccaatcctcaaaaataaaatggcccttccaaaattagagacatatcgtagaaatcagacgcacgcttggcgtcaatatcctaaaacggaaaactaatcagttagatgtgtcttttttaggtaaaaaaaggcaaccaactttttgctggctagcgatgcgacaataaacgcgcgcagc

SEQ ID NO: 140 - DNA, 1440 bp, synthetic construct
  variation 856..866: note = n is a, c, g, or t
  source 1..1440: mol_type = other DNA
gggccaatttgttggagtccggatcatccaaatgcgctaatatcttcttcgtaaatgtggccgttatttgtgcttgatttgcgtcattcattaccatagtattactattggtgttcatatttctgttattactgttattagtttggcgttttgcctgacgatcagcctggtggggccaagtatatactataaaaacaggttagttacttgctctatgcgtttgcgcatcctctttttactttttttttttcagtaaagcctaagcataaatcgttttatacgtacgacacgttcaacttttcttggttagtagtggcaatctctgcaatacatacagggagtcatggtctatcatcttgtccaatcaaagaagcatcggttcagatcgagcaaactgtagggagaaaggaaagtagaaatgcagagtgtgctatatgtccaatctcggttttgtagtttggatgtcattagagatctaccacccaaccggctgctttcatgtggaacagaaaagaaatcggggcgcttcctcttctgtattcctttaattaacgtttttattcagccatctaaccatcatacccccatacggtaacaaaacctcttctaagaaaagaagtctctgctcctccgccatcttatttttattcgctgcgcgcgtttattgtcgcatcgctagccagcaaaaagttggttgcctttttttacctaaaaaagacacatctaactgattagttttccgttttaggatattgacgccaagcgtgcgtctgatttctacgatatgtctctaattttggaagggccattttatttttgaggattgggctgatcattttccttacgtggattgagnnnnnnnnnnngcaatacagatcattattaaactgttttgtacatgatgttagtatataatcgtaaagcttttctaatatgtataccttatacatggaactccacagaacttgcaaacataccaaaaatcctttattcttgttcactcattttacatcaaaaaataatatttcagttattaaggaaaataaaaaaatagattagagaagcattttgaagaaatagtatattcttttattgaacctaagagcgtgatatttttactcgaaataaaatacgaaaaatctatacactcatctttccgactactattggctcctgctcaaaaaaagagggaaaaaaagctccaaaattctatcttttcctatcgctcctgtcctatccttattacgttcattactattttaatactatccattcttttattttcagtctaaaaaaaacatttctcataacgggaaaagcaaaaaaatgtcaagcttatacatcaaaacaccactgcatgcattatctgctggtccggattctcatgcaaatagctcctattatgacaatcttttattgccttcattttccaatttatcc

SEQ ID NO: 141 - DNA, 1440 bp, synthetic construct
  variation 575..585: note = n is a, c, g, or t
  source 1..1440: mol_type = other DNA
ggataaattggaaaatgaaggcaataaaagattgtcataataggagctatttgcatgagaatccggaccagcagataatgcatgcagtggtgttttgatgtataagcttgacatttttttgcttttcccgttatgagaaatgttttttttagactgaaaataaaagaatggatagtattaaaatagtaatgaacgtaataaggataggacaggagcgataggaaaagatagaattttggagctttttttccctctttttttgagcaggagccaatagtagtcggaaagatgagtgtatagatttttcgtattttatttcgagtaaaaatatcacgctcttaggttcaataaaagaatatactatttcttcaaaatgcttctctaatctatttttttattttccttaataactgaaatattattttttgatgtaaaatgagtgaacaagaataaaggatttttggtatgtttgcaagttctgtggagttccatgtataaggtatacatattagaaaagctttacgattatatactaacatcatgtacaaaacagtttaataatgatctgtattgcnnnnnnnnnnnctcaatccacgtaaggaaaatgatcagcccaatcctcaaaaataaaatggcccttccaaaattagagacatatcgtagaaatcagacgcacgcttggcgtcaatatcctaaaacggaaaactaatcagttagatgtgtcttttttaggtaaaaaaaggcaaccaactttttgctggctagcgatgcgacaataaacgcgcgcagcgaataaaaataagatggcggaggagcagagacttcttttcttagaagaggttttgttaccgtatgggggtatgatggttagatggctgaataaaaacgttaattaaaggaatacagaagaggaagcgccccgatttcttttctgttccacatgaaagcagccggttgggtggtagatctctaatgacatccaaactacaaaaccgagattggacatatagcacactctgcatttctactttcctttctccctacagtttgctcgatctgaaccgatgcttctttgattggacaagatgatagaccatgactccctgtatgtattgcagagattgccactactaaccaagaaaagttgaacgtgtcgtacgtataaaacgatttatgcttaggctttactgaaaaaaaaaaagtaaaaagaggatgcgcaaacgcatagagcaagtaactaacctgtttttatagtatatacttggccccaccaggctgatcgtcaggcaaaacgccaaactaataacagtaataacagaaatatgaacaccaatagtaatactatggtaatgaatgacgcaaatcaagcacaaataacggccacatttacgaagaagatattagcgcatttggatgatccggactccaacaaattggccc